(19) United States
(12) Patent Application Publication
(10) Pub. No.: US 2024/0169540 A1
(43) Pub. Date: May 23, 2024

(54) METHOD AND SYSTEM FOR QUANTITATIVE MICROVASCULAR DYSFUNCTION ON SEQUENCES OF ANGIOGRAPHIC IMAGES

(71) Applicant: Pie Medical Imaging B.V., Maastricht (NL)

(72) Inventors: Chris Bouwman, Oirsbeek (NL); Dennis Koehn, Voerendaal (NL); Jean-Paul Aben, Limbricht (NL)

(73) Assignee: Pie Medical Imaging B.V., Maastricht (NL)

(21) Appl. No.: 18/516,286

(22) Filed: Nov. 21, 2023

Related U.S. Application Data
(60) Provisional application No. 63/384,860, filed on Nov. 23, 2022.

(51) Int. Cl. G06T 7/00; G06T 7/10; G06T 7/246; G06T 7/62; G06T 7/70

(52) U.S. Cl. CPC G06T 7/0016; G06T 7/10; G06T 7/248; G06T 7/62; G06T 7/70; G06T 2207/10116; G06T 2207/20081; G06T 2207/30048; G06T 2207/30104

(57) ABSTRACT

Computer-implemented methods and systems are provided for charactering a property of microvascular tissue that is supplied with blood via a coronary artery under investigation, which involve obtaining an x-ray angiographic image sequence of the coronary artery under investigation acquired while contrast agent flows into and through the coronary artery under investigation. The angiographic image sequence is used to determine a volumetric flow rate for flow through the coronary artery under investigation, which is used to determine an index that represents a property of the microvascular tissue that is supplied with blood via the coronary artery under investigation.

### CROSS-REFERENCE TO RELATED APPLICATION(S)

The present application claims priority from U.S. Prov. Appl. No. 63/384,860 entitled “METHOD AND SYSTEM FOR QUANTITATIVE MICROVASCULAR DYSFUNCTION ON SEQUENCES OF ANGIOGRAPHIC IMAGES,” filed on Nov. 23, 2022, herein incorporated by reference in its entirety.

### BACKGROUND

### 1. Field

The present disclosure relates to methods and systems that capture and analyze angiographic images for assessment of coronary artery disease.

### 2. State of the Art

Coronary artery disease (CAD) is one of the leading causes of death and serious illness in the Western world. Patients suffering from CAD experience angina pectoris being the most common symptoms of CAD, which affects approximately 112 million people globally. The 2019 ESC guidelines (Knuuti, Juhani et al. “2019 ESC Guidelines for the diagnosis and management of chronic coronary syndromes”, European heart journal vol. 41,3 (2020): 407-477) provides guidance on the diagnosis and management of patients with chronic CAD. However, a large proportion (around 50%) of the patients with symptoms of angina pectoris undergoing coronary angiography do not have obstructive coronary artery disease but have demonstratable ischemia and many of these patients have a normal or near normal coronary angiogram, presenting a diagnostic challenge to clinician. This condition is called coronary microvascular dysfunction which refers to a dysfunction in the small vessels (micro vessels) of the heart, with focus on the coronary circulation. Coronary microvascular dysfunction primarily affects the arterioles and capillaries of the heart. These vessels are responsible for regulating blood flow within the heart muscle and ensuring that the heart receives enough oxygen and nutrients. Coronary microvascular dysfunction is often considered an important problem in these patients with so called angina and no obstructive coronary artery disease (ANOCA). Coronary microvascular dysfunction is found more commonly in females compared to males (Merz, C N et al. “The Women's Ischemia Syndrome Evaluation (WISE) study: protocol design, methodology and feasibility report”, Journal of the American College of Cardiology vol. 33,6 (1999): 1453-61). Coronary microvascular dysfunction is an area of active research in cardiology, and understanding of its causes, diagnosis, and management is evolving. It is important for individuals experiencing symptoms suggestive of heart disease to seek medical attention, and for healthcare professionals to consider coronary microvascular dysfunction as a potential cause, especially in cases where traditional tests do not provide a clear diagnosis.

Patients suffering from CAD disease are primarily treated by performing a percutaneous coronary intervention (PCI), which is a is a non-surgical procedure, performed in the catheterization laboratory using X-ray angiography, that uses a catheter (a thin flexible tube) to place a small structure called a stent to open up blood vessels in the heart that have been narrowed by plaque buildup, a condition known as atherosclerosis. This assumes that the angina pectoris symptoms are caused by narrowing of a coronary artery resulting in impedes oxygen delivery to the heart muscle (myocardium). As indicated above a huge number of patients with symptoms of myocardial ischemia (angina pectoris) have no obstructive CAD and Coronary microvascular dysfunction is often unrecognized and undertreated (Bairey Merz, C Noel et al. “Ischemia and No Obstructive Coronary Artery Disease (INOCA): Developing Evidence-Based Therapies and Research Agenda for the Next Decade”, Circulation vol. 135,11 (2017): 1075-1092).

Nowadays, within the catheterization laboratory the interventional cardiologists can use bolus thermodilution or continuous thermodilution to assess microvascular dysfunction (Candreva, Alessandro et al. “Basics of Coronary Thermodilution”, JACC. Cardiovascular interventions vol. 14,6 (2021): 595-605). Both techniques require the insertion of a wire with a pressure sensor and temperature sensor into the coronary artery and inducing hyperemia. The index of microvascular resistance (IMR) is an established method to assess microvascular disease and is based on an invasive bolus thermodilution measurement. IMR is a dimensionless index and is calculated by multiplying the distal coronary pressure with the mean transit time during maximal hyperemia, both measured by insertion of a wire in the coronary artery which measures the pressure and difference in temperature after injection of cold saline to extract the mean transit time. Another invasive approach is based on Doppler pressure wire and measures the hyperemic microvascular resistance (HMR). HMR is defined as the distal pressure divided by simultaneously measured flow velocity during hyperemia (Meuwissen, M et al. “Role of variability in microvascular resistance on fractional flow reserve and coronary blood flow velocity reserve in intermediate coronary lesions”, Circulation vol. 103,2 (2001): 184-7). However, these techniques have not been widely incorporated into routine practice due to technical challenges, procedural costs, increased procedure time, and the intolerance some patients have to hyperemia.

### SUMMARY

In embodiments herein, computer-implemented methods and systems are described that characterize a property of microvascular tissue that is supplied with blood via a coronary artery under investigation, which involve:
i) obtaining an x-ray angiographic image sequence of the coronary artery under investigation acquired while contrast agent flows into and through the coronary artery under investigation;
ii) using the angiographic image sequence of i) to determine a volumetric flow rate for flow through the coronary artery under investigation; and
iii) determining an index that represents a property of the microvascular tissue that is supplied with blood via the coronary artery under investigation based on the volumetric flow rate of ii).

In embodiments, the operations of i) to iii) can be performed automatically by a processor without human input.

In embodiments, the volumetric flow rate can be based on flow velocity of a contrast bolus front within the angiographic image sequence of i) and cross-sectional area of the coronary artery under investigation at multiple positions along the coronary artery under investigation within the angiographic image sequence of i).

In embodiments, the volumetric flow rate can be based on propagation time of a contrast bolus front within the angiographic image sequence of i) and a vessel volume for the coronary artery of interest.

In embodiments, the vessel volume can be determined from a 3D reconstruction of the coronary artery of interest.

In embodiments, the vessel volume can be based on determining one or more diameters of the coronary artery of interest along the of the coronary artery of interest.

In embodiments, the flow velocity of the contrast bolus front can be determined from distance that the contrast bolus front travels in the angiographic image sequence of i) as a function of time.

In embodiments, the flow velocity of the contrast bolus front can be determined from image analysis of the angiographic image sequence of i), wherein the image analysis determines a proximal position for the coronary artery of interest, a distal position for the coronary artery of interest, a vessel path extending along the coronary vessel of interest between the proximal position to the distal position, and propagation of the contrast bolus front along the vessel path.

In embodiments, at least one of the proximal position and the distal position can be determined using artificial intelligence and/or deep learning techniques.

In embodiments, the artificial intelligence and/or deep learning techniques can employ dichotomous image segmentation.

In embodiments, the artificial intelligence and/or deep learning techniques can employ additional information selected from the groups consisting of vessel type, rotation and angulation used in image acquisition, ECG information, heart dominance information, and time between image frames.

In embodiments, the artificial intelligence and/or deep learning techniques can employ a vesselness filter applied to multiple image frames of the angiographic image sequence of i).

In embodiments, the proximal position can be determined from detection of position of a guiding catheter used for injection of the contrast agent into the coronary vessel of interest.

In embodiments, the vessel path can be determined using a wave propagation algorithm between the proximal position and distal position.

In embodiments, the microvascular tissue can be part of the myocardium.

In embodiments, the volumetric flow rate of ii) is characteristic of volumetric flow rate for part of a cardiac cycle.

In embodiments, the volumetric flow rate of ii) is characteristic of average flow velocity and average volumetric flow rate over a cardiac cycle.

In embodiments, the index can include quantitative data that represents amount of dysfunction or resistance in the microvascular tissue that is supplied with blood via the coronary artery under investigation.

In embodiments, the index can be determined from the volumetric flow rate of ii) and determination of a pressure drop associated with the coronary artery under investigation;

In embodiments, the index can be normalized based on at least one parameter selected from the group consisting of cardiac mass, coronary volume, coronary artery cross-sectional area, patient weight, height, body surface area (BSA) or body mass index (BMI), heart dominance, or combinations thereof.

In embodiments, the index can include quantitative data that represents the ratio of flow through the coronary artery under investigation at rest relative to flow through the coronary artery under investigation in the hyperemic state.

In embodiments, the method can involve using the at least one angiographic image of i) to determine a first volumetric flow rate for flow through the coronary artery under investigation with the patient in a rest state, using the at least one angiographic image of i) to determine a second volumetric flow rate for flow through the coronary artery under investigation with the patient in an active/hyperemic state, and determining the index from the first and second volumetric flow rates.

In another aspect, a non-transitory computer readable medium can be provided that has instructions stored thereon, wherein the instructions can be executed by a computing device to cause the computing device to perform the methods as described herein to characterize a property of microvascular tissue that is supplied with blood via a coronary artery under investigation.

In yet another aspect, an imaging system can be provided that includes a data processor configured to perform the methods as described herein to characterize a property of microvascular tissue that is supplied with blood via a coronary artery under investigation.

Other aspects are described and claimed.

### BRIEF DESCRIPTION OF THE DRAWINGS

The characteristics of the invention and the advantages derived therefrom will be more apparent from the following description of non-limiting embodiments, illustrated in the annexed drawings, in which:

FIG. 1 shows a flow chart of a method for determining microvascular dysfunction in accordance with an embodiment herein.

FIG. 2 shows a functional block diagram of an exemplary single plane angiographic system.

FIG. 3A shows a functional block diagram of an exemplary method that calculates coronary volumetric flow rate based on contrast bolus velocity and vessel area.

FIG. 3B is an example X-ray angiographic image that can be processed as part of the method of FIG. 3A to calculate coronary volumetric flow rate.

FIG. 4A illustrates an exemplary method that calculates coronary flow velocity from an X-ray angiographic image sequence.

FIG. 4B is an example X-ray angiographic image that can be processed as part of the method of FIG. 4A to calculate coronary flow velocity.

FIGS. 5A1 to 5A4 show an illustration of tracking the contrast bolus front within an X-ray angiographic image sequence.

FIG. 5B1 shows the coronary centerline of a vessel of interest after identifying proximal location and distal location for the vessel of interest.

FIG. 5B2 shows an example of a tracked centerline of a vessel of interest.

FIG. 6 shows an example of a screenshot of CAAS Workstation Bolus tracking, in which the disclosed embodiments is implemented.

FIG. 7 shows a flow chart of a method for fully automated initiation of the contrast bolus tracking in accordance with an embodiment herein.

FIG. 8 shows an illustration of the fully automatic centerline determination.

FIG. 9 provides an illustration of another method to determine the coronary velocity.

FIG. 10A shows a functional block diagram illustrating a method that calculates coronary volumetric flow rate based on contrast bolus propagation time and vessel volume.

FIG. 10B is an example X-ray angiographic image that can be processed as part of the method of FIG. 10A to calculate coronary volumetric flow rate.

FIG. 11 shows an example of a QCA3D.

FIG. 12 shows a parabolic coronary velocity profile.

FIG. 13 illustrates that during one cardiac cycle the coronary volumetric flow rate and flow velocity is not constant.

FIG. 14 provides an illustration of the pressure drop within the coronary system.

FIG. 15 illustrates a method the defined the myocardial mass or volume from X-ray angiography.

FIG. 16 illustrated another method to determine the myocardial mass from X-ray angiography.

FIG. 17 illustrates an example of a high-level block diagram of an X-ray cinefluorograpic system.

FIG. 18 shows the definitions according to a general model for the coronary tree according to the American Heart Association.

FIG. 19 illustrates the different patient states and options to extract the flow within different patient states.

FIG. 20A illustrates an alternative approach to determine the contrast bolus propagation time.

FIG. 20B is an example X-ray angiographic image that can be processed as part of the method of FIG. 20A to determine the contrast bolus propagation time.

FIG. 21A illustrates a different approach to evaluate myocardial status without determination of contrast velocity or contrast bolus transit time.

FIG. 21B is an example X-ray angiographic image that can be processed as part of the method of FIG. 21A to evaluate myocardial status.

FIG. 22 illustrates the difference in the cross section of micro vessels for normal microcirculation, structural microvascular dysfunction and functional microvascular dysfunction, for the case of the microvascular vessel at rest and the case of the microvascular vessel under stress.

### DETAILED DESCRIPTION OF THE PREFERRED EMBODIMENTS

The present disclosure describes method(s) and system(s) to assess microvascular dysfunction using X-ray angiographic image data.

The present disclosure relates to method(s) and system(s) that quantify microvascular dysfunction based on two-dimensional (2D) X-ray angiographic image data and it will be mainly disclosed with reference to this field.

As used herein, the term “image” or “image frame” refers to a single image, and the term “image sequence” ort “image data” can refer to multiple images acquired over time and when used in relation to X-ray imaging it comprises multiple image frames covering one or more phases of the cardiac cycle.

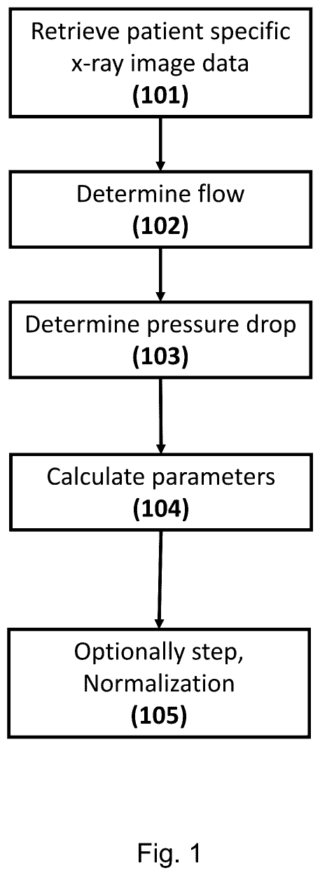

FIG. 1 shows a flow chart illustrating the operations according to an embodiment of the present application. The operations employ an imaging system capable of acquiring and processing one or more two-dimensional X-ray angiographic image sequences of a vessel organ (or portion thereof) or other object of interest. For example, a single plane or bi-plane angiographic system can be used to acquire the one or more X-ray angiographic image sequences. Examples of such systems are those manufactured by Siemens (Artis zee Biplane) or Philips (Allura Xper FD).

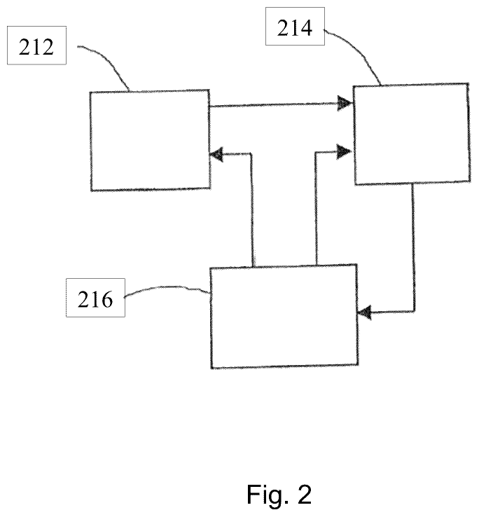

FIG. 2 is a functional block diagram of an exemplary single plane angiographic system, which includes an angiographic imaging apparatus 212 that operates under commands from user interface module 216 and will provide data to data processing module 214. The single plane angiographic imaging apparatus 212 captures a two-dimensional X-ray image sequence of the vessel organ of interest for example in the posterior-anterior direction. The single plane angiographic imaging apparatus 212 typically includes an X-ray source and detector pair mounted on an arm of a supporting gantry. The gantry provides for positioning the arm of the X-ray source and detector at various angles with respect to a patient who is supported on a table between the X-ray source and detector. The data processing module 214 may be realized by a personal computer, workstation, or other computer processing system. The data processing module 214 processes the two-dimensional image sequence captured by the single plane angiographic imaging apparatus 212 to generate data as described herein. The user interface module 216 interacts with the user and communicates with the data processing module 214. The user interface module 216 can include different kinds of input and output devices, such as a display screen for visual output, a touch screen for touch input. a mouse pointer or other pointing device for input, a microphone for speech input, a speaker for audio output, a keyboard and/or keypad for input, etc. The data processing module 214 and the user interface module 216 cooperate to carry out the operations of FIG. 1 as described below.

The operations of FIG. 1 can also be carried out by software code that is embodied in a computer product (for example, an optical disc or other form of persistent memory such as a USB drive or a network server). The software code can be directly loadable into the memory of a data processing system for carrying out the operations of FIG. 1. Such data processing systems can also be physically separated from the angiographic system used for acquiring the images making use of any type of data communication for getting such images as input.

In this example, it is assumed that the X-ray imaging system has acquired and stored at least one two-dimensional image sequence of an object of interest. Any image device capable of providing two-dimensional angiographic image sequences can be used for this purpose. For example, a bi-plane or single plane angiographic system can be used. Examples of such systems are those manufactured by Siemens (Artis zee Biplane) or Philips (Allura Xper FD).

An embodiment is now disclosed with reference to FIG. 1. The operations depicted in FIG. 1 can be performed in any logical sequence and can be omitted in parts. As it is an objective of the application to provide a select (e.g., optimal) workflow that can be used during the interventions, workflow example steps will also be referenced.

As can be seen in FIG. 1, the workflow comprises of a number of steps. The first step (101) of FIG. 1 involves the retrieval of a patient specific X-ray angiographic image sequence. The workflow of the present disclosure is based on the filling of the coronary vessels with a contrast agent (or “contrast liquid”); therefore, the acquisition of the angiographic image data can start before contrast injection of the contrast agent and continues while filling of the coronary vessel of interest with the contrast agent until it is filled with the contrast agent its entirety and can include the wash out of contrast liquid of the coronary vessel.

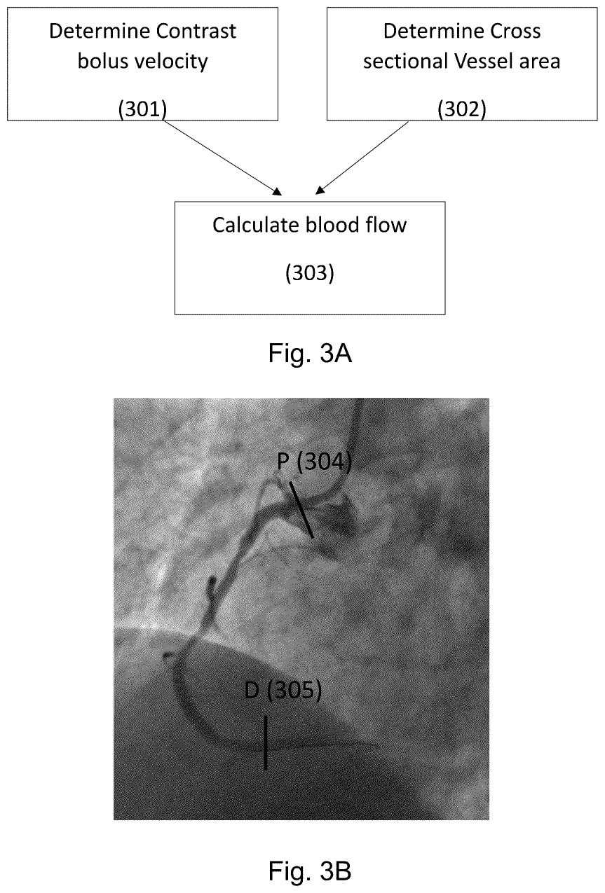

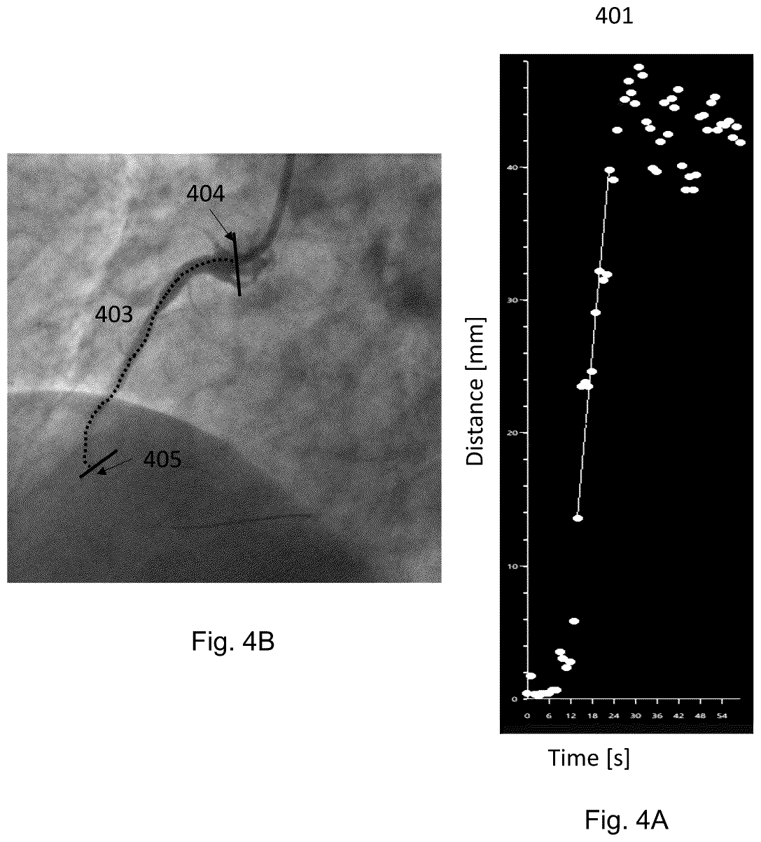

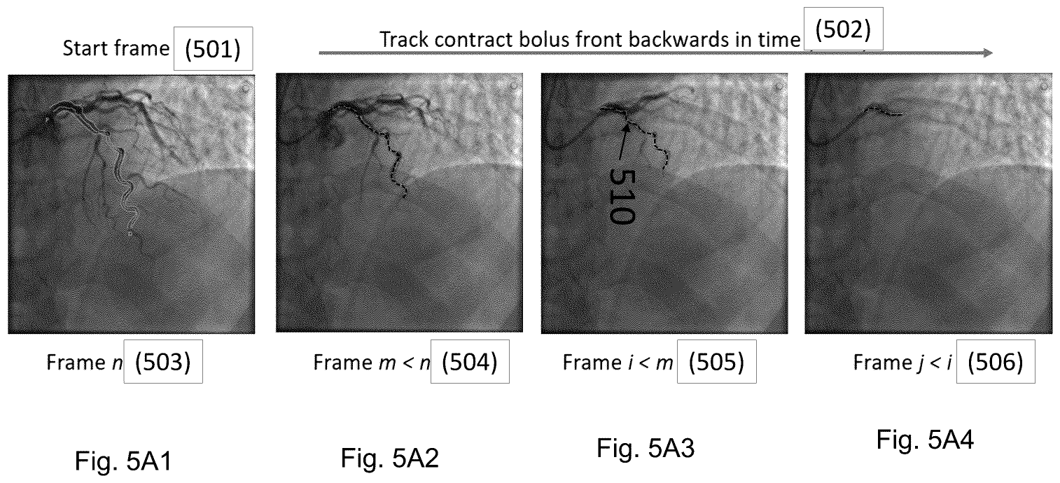

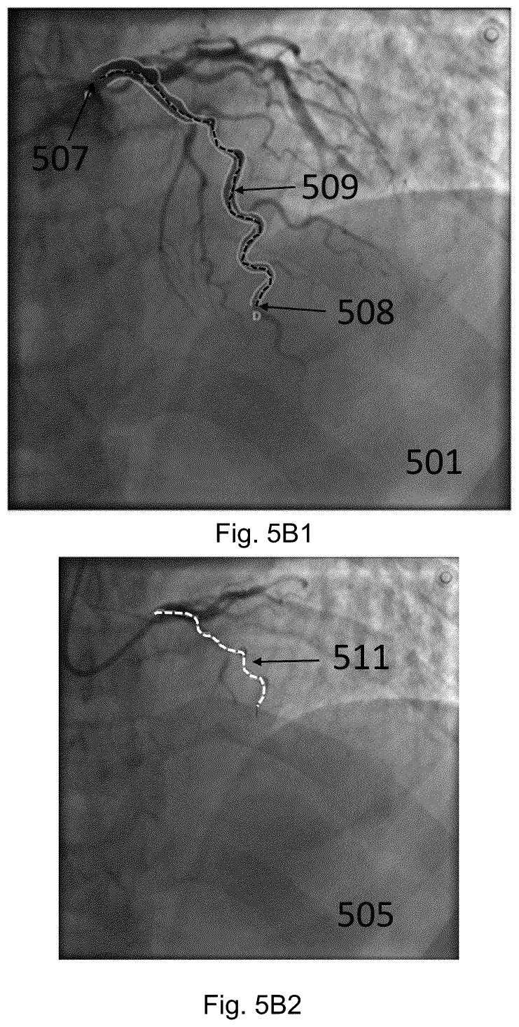

Within step 102 of FIG. 1, coronary flow is derived from the X-ray image sequence retrieved in step 101. The coronary volumetric flow rate (303) can be calculated using the flow velocity of the contrast bolus front within the X-ray image sequence (301) multiplied with the cross-sectional area of the vessel (302) as shown by the flowchart of FIG. 3A. The flow velocity of the contrast bolus within the X-ray image data will represent the coronary flow velocity. The coronary flow velocity can be derived from X-ray angiography as explained by Zhang, Yimin et al. “Automatic coronary blood flow computation: validation in quantitative flow ratio from coronary angiography”, the international journal of cardiovascular imaging vol. 35,4 (2019): 587-595. Within each frame of the contrast injection period, the distance of the contrast travelled (403) can be plotted against the time (401) as shown in FIG. 4A. Within FIG. 4A (401), the x-axis represents the frame (time) within the X-ray angiographic image sequence, and the y-axis represents the distance from the proximal start position (404) till the contrast bolus within a particular frame of the X-ray angiographic image sequence, for instance as illustrated by 405 in FIG. 4B. As an X-ray angiographic image sequence is acquired during contrast injection, the latter frames within the X-ray angiographic image sequence will result in visualization of the more distal vessel location (see also FIG. 5A1 to 5A4), resulting in a longer distance as can be seen in graph 401. FIGS. 5A1 to 5A4 show an illustration of tracking the contrast bolus front within an X-ray angiographic image sequence. Image 501 of FIG. 5A1 represents the start frame. A centerline, representing the coronary vessel segment of interest, is required to start the bolus tracking. This centerline can for instance be derived from a coronary segmentation as described by Gronenschild et al. “CAAS. II: A second generation system for off-line and on-line quantitative coronary angiography”, catheterization and cardiovascular diagnosis vol. 33,1 (1994): 61-75 or manually identified. FIG. 5B1 shows the coronary segmentation of a vessel of interest after identifying the proximal location (507) and distal location (508) and the resulting centerline (509) after coronary segmentation in the start frame 501 of FIG. 5A1. FIGS. 5A2 to 5A4 shows an illustration of tracking the contrast bolus front backwards in time relative to the start frame of FIG. 5A1. Image 504 of FIG. 5A2 is a frame earlier in the image sequence relative to the start frame (501). Image 505 of FIG. 5A3 is a frame earlier in the image sequence relative to frame 504. Image 506 of FIG. 5A4 is a frame earlier in the image sequence relative to frame 505. Furthermore, the tracked centerline (from proximal location 507 in this sequence is shown as 510 in FIG. 5A3 and 511 in FIG. 5B2.

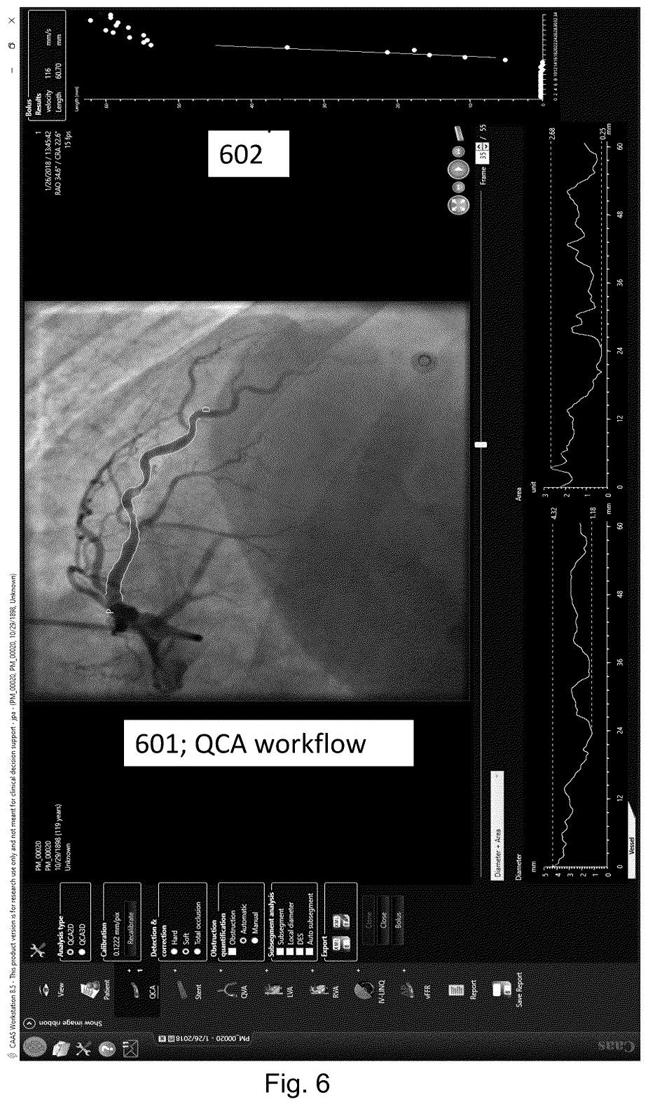

Referring to FIG. 4A, the upslope (402) of the distance of the contrast bolus front travelled plotted against the time (401), represents the contrast bolus propagation. By fitting a line (402), e.g., linear, through this upslope, the coronary flow velocity can be determined, or using for example the first derivative of the graph (401). FIG. 6 shows an example of a screenshot of CAAS Workstation Bolus tracking, in which the method above is implemented within the QCA (quantitative coronary analysis) workflow (601); the bolus tracking is performed after QCA segmentation, and the graph representing the distance of the contrast bolus front travelled plotted against the time is shown (602). The initial centerline can be extracted from the QCA segmentation. In case a 3D coronary reconstruction has been performed, the coronary velocity can be derived using both projections which were used to create the 3D coronary reconstruction. The 3D based coronary velocity can be derived by computing the average of the coronary velocity obtained by each projection in accordance with the methods described in this patent application. A weighted average is also possible in which the weights are based on the foreshortening of each projection used to create the 3D coronary reconstruction.

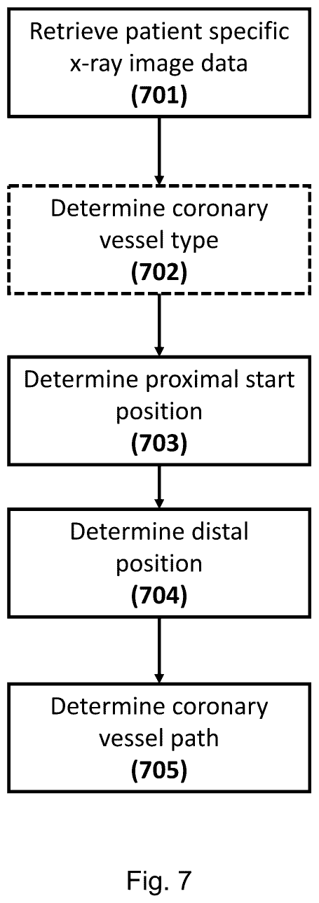

Within a preferred embodiment, the initiation of the contrast bolus tracking as described with reference to FIGS. 4A, 4B, 5A1 to 5A4, 5B1, 5B2 and 6, is performed fully automatically without any human interaction. This will allow calculation of coronary blood flow and/or microvascular resistance directly after the acquisition of the X-ray angiographic image data in accordance with the methods described by this patent application. This means that the physician received information of the patient specific coronary blood flow and/or microvascular resistance directly after the image acquisition within the catheterization room. As during a PCI and/or diagnostic coronary angiography, multiple X-ray angiographic image acquisitions are performed, the physician received a good impression of the coronary blood flow and/or microvascular resistance during the procedure which allows the physician to provide better treatment choices/options. The method for fully automated initiation of the contrast bolus tracking will be described with reference to the flowchart of FIG. 7.

At step 701, the patient specific X-ray image data is received and is similar to step 101 of the flowchart from FIG. 1. Coronary angiography, also known as cardiac catheterization or coronary arteriography, is a medical procedure used to visualize the coronary arteries, which supply blood to the heart muscle. X-ray coronary angiography involves the use of X-ray imaging technology to create detailed images of the coronary arteries. The contrast liquid is injected using a catheter which is either placed in the left coronary ostium or the right coronary ostium. Injections of contrast liquid in the left coronary ostium, results in visualization of the LAD and LCX, while injections of contrast liquid in the right coronary ostium results in the visualization of the right coronary artery. To distingue between the three major coronary vessels (RCA, LAD, LCX), within step 702 of the flowchart from FIG. 7, the coronary vessel type is determined based on the image data (image sequence) from step 701. Although step 702 is an optional step, it may provide additional information for the physician, for instance for reporting, and additional input for the following workflow step within FIG. 7. The coronary vessel type can be determined by an artificial intelligence classification network trained to recognize within an X-ray image frame or sequence the dominance presents of a RCA, LAD and LCX coronary artery. Such a classification network can be deployed using deep learning networks or deep convolutional neural networks. For instance, the deep learning methods as described by Serife Kaba et al. in “The application of deep learning for the segmentation and classification of coronary arteries”, Diagnostics 2023, 13, 2274. Optionally, the C-arm rotation and angulation can be used as additional information to improve the performance of such artificial intelligence methods.

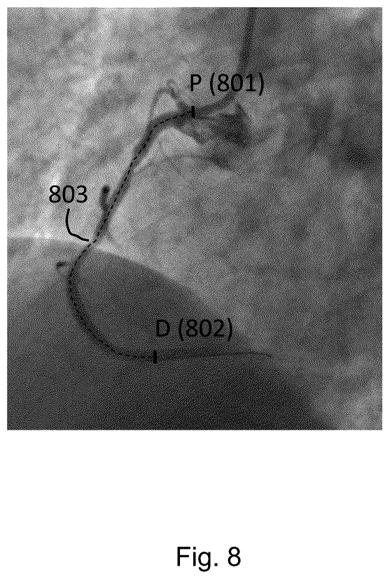

At step 703, the proximal start position is determined. The proximal start position represents the ostium of the coronary artery, and this will be either the ostium of the right coronary artery or the left coronary artery due to the way the contrast is injected as described above. Within FIG. 8 an illustration is provided of the fully automatic centerline determination. The proximal start position, identified by “P” (801) within FIG. 8, can be determined by using for instance artificial intelligence and/or deep learning techniques. An example of a deep learning technique to determine the proximal start position (801) is by using the IS-Net proposed by Qin et at., “Highly Accurate Dichotomous Image Segmentation”, Computer Vision—ECCV 2022 (2022): 38-56. Within the work disclosed by Qin et at. objects within an image are detected through dichotomous image segmentation. Dichotomous image segmentation is a type of image segmentation method that involves dividing an image into two distinct regions or classes. The word “dichotomous” itself refers to the division of something into two parts. In image segmentation, the goal is to partition an image into meaningful and homogeneous regions based on certain characteristics or criteria. The process of dichotomous image segmentation typically involves distinguishing between two classes or regions in an image, often representing objects or background, foreground or background, or different types of objects. The segmentation is performed based on certain features such as intensity, color, texture, or other visual properties. By training the IS-Net proposed by Qin et at. in detecting the ostium of the left coronary artery and right coronary artery the proximal start position can be determined. Extra input that can be used is vessel type (from step 702) and/or information such as the rotation and angulation of the c-arm to guide the neural network towards optimal landmarks for those specific rotation and angulation angles. Other information that could be provided is information regarding ECG, heart dominance, time between image frames, etc. Another method to determine the proximal start position is to detect the guiding catheter in the image. As the guiding catheter is used to inject the contrast liquid either in the left coronary artery or the right coronary artery, the guiding catheter tip will represent the proximal start position. An example of a method to detect the guiding catheter tip is disclosed by U.S. Pat. No. 11,707.242 “Method and system for dynamic coronary roadmapping”, in which a catheter tip detection and tracking method is described by use of a deep learning-based Bayesian filtering method. The described method in U.S. Pat. No. 11,707,242 models the likelihood term of Bayesian filtering with a convolutional neural network, and integrates it with particle filtering in a comprehensive manner, leading to more robust catheter tip detection and tracking. In summary, for every position within an image, the new position (in new image or new frame within the image sequence) of the catheter tip (predict movement of catheter) can be predicted using the optical flow method and the addition of noise. Further, update the weight by checking the likelihood of the position using the deep learning network. Next, all weights are normalized. The real catheter tip position equals the weighted arithmetic mean of all positions and their weights. Finally, a resample of points is performed around the position with a high weight value. Alternatively, the proximal start position can be determined by conventional image processing techniques, such as template matching either by using a template of the catheter tip, or a template of the coronary ostium. Another approach is by performing a vesselness filter on multiple frames within the image sequence as for instance as proposed by Frangi et al., “Multiscale vessel enhancement filtering”, Medical Image Computing and Computer-Assisted Intervention—MICCAI'98 (1998): 130-137. Next. process the output of the vesselness filter to determine the start location, for instance by artificial intelligence techniques.

At step 704 of FIG. 7, the distal position is determined. Within FIG. 8 an example of a distal position is provided by 802. The distal position can be automatically found using artificial intelligence/deep learning. The artificial intelligence network can use an angiographic image frame or an angiographic image sequence as input. The network can find landmarks that are located at the distal side of a coronary vessel. If multiple coronary vessels are visible within the image frame, a distal position (802) can be found for each of those, which would be the case in a left coronary angiogram as the LAD and LCX are visible. An example of an artificial intelligence model that is able to detect these landmarks is IS-Net proposed by Qin et at., “Highly Accurate Dichotomous Image Segmentation”, Computer Vision—ECCV 2022 (2022): 38-56. Extra input that can be used is vessel type (from step 1402) and/or information such as the rotation and angulation of the c-arm to guide the neural network towards optimal landmarks for those specific rotation and angulation angles. Other information that could be provided is information regarding ECG, heart dominance, time between image frames, etc. Another method to accomplish finding a distal location (802) is by processing the image directly. An example of finding the distal position is by performing a vesselness filter on multiple frames within an image sequence and tracking the changes of the filter output. Such a filter is proposed by Frangi et al., “Multiscale vessel enhancement filtering”, Medical Image Computing and Computer-Assisted Intervention—MICCAI'98 (1998): 130-137.

At step 705 of FIG. 7, the coronary vessel path (803) is determined by using the proximal start position (as a result of step 703) and the distal position (as a result of step 704). The coronary vessel path can be determined by for instance using CAAS Workstation 8.5, QCA workflow (Pie Medical Imaging, the Netherlands) using the determined proximal start position and distal position. Another method to determine the coronary vessel path is by means of a wave propagation algorithm between the determined proximal start position and distal position. An example of a wave propagation algorithm is described by Janssen et al., “A novel approach for the detection of pathlines in X-ray angiograms: the wavefront propagation algorithm”, Int J Cardiovasc Imaging 2002; 18(5):317-324. Another method to determine the coronary vessel path (803) is by first determining all vessels within the image and hereafter selecting the segment between the proximal and distal marker. Such a determination can for example be performed by a machine learning network as proposed by Zhang et al., “X-ray coronary centerline extraction based on C-UNet and a multifactor reconnection algorithm”, Computer Methods and Programs in Biomedicine (2022) 226. 107114.

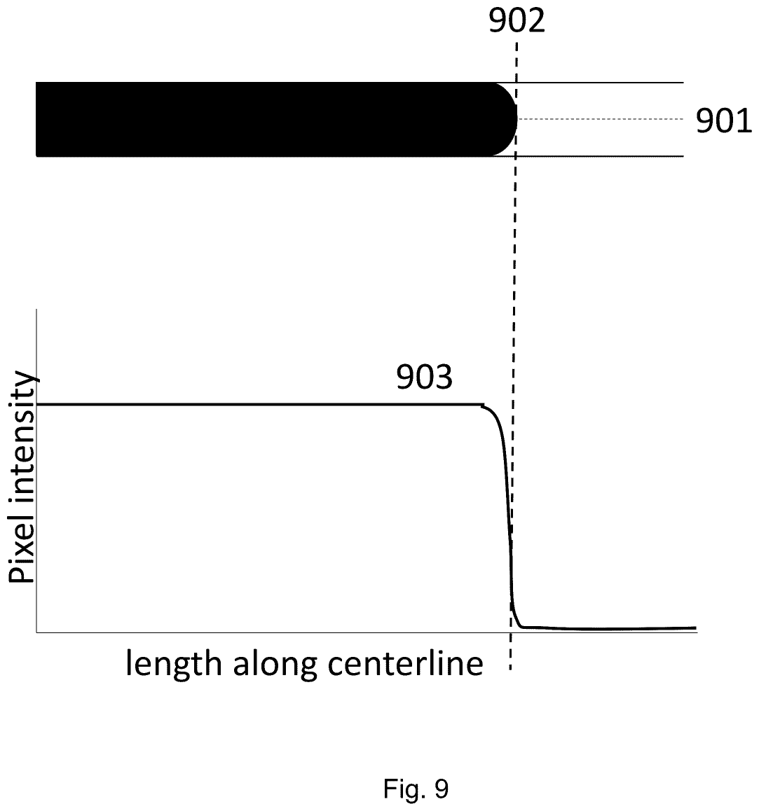

Referring back to the description of 102 of FIG. 1, another approach to determine the coronary velocity is explained in U.S. patent application Ser. No. 15/971,275 “Method and Apparatus for determining blood velocity in X-ray angiographic images” in which the pixel intensity profiles (FIG. 9, 903) along the centerline (FIG. 9, 901) of the vessel in two different image frames in time are analyzed. The intensity will change at the contrast bolus front (FIG. 9, 902) and can be detected by the disclosed method within U.S. patent application Ser. No. 15/971,275. The length and time difference between the two frames can be used to determine the coronary flow velocity.

To convert the coronary flow velocity (301) into coronary volumetric flow rate (303), it must be multiplied with the cross-sectional area (302) as summarized in FIG. 3A. This can be done by multiplying the cross-sectional area at each position along the vessel path (between 304 and 305) with the coronary flow velocity as depicted in FIG. 3B, and integrating the resultant products over the number of positions along the vessel length as specified in Eqn. (1). The advantage of integrating the cross-sectional areas of the vessel part of interest, is that in case of bifurcations the coronary volumetric flow rate reduces after the bifurcation, and this is taken into account by the reduction in cross-sectional area after the bifurcation. The cross-sectional area can be determined using for example by using for the QCA3D further describe by Girasis et al. in “Advanced three-dimensional quantitative coronary angiographic assessment of bifurcation lesions: methodology and phantom validation”, EuroIntervention 8:1451-1460, 2013, or QCA workflow within CAAS Workstation 8.5 (Pie Medical Imaging, the Netherlands), or using densitometry for area calculations or assuming circularity as described by Gronenschild et al. “CAAS. II: A second generation system for off-line and on-line quantitative coronary angiography”, catheterization and cardiovascular diagnosis vol. 33,1 (1994): 61-75.

$$Q = {\sum }_{i = 1}^{i = n} \frac{{A}_{i} * v}{n} \qquad Eqn . ( 1 )$$

Q=coronary volumetric flow rate
Aᵢ=Cross-sectional area at each position along the coronary artery
v=coronary flow velocity
n=number of cross-sectional area values along the coronary artery

The coronary flow velocity can also be determined manually. This can be done by dividing the length of a vessel segment by the time it takes for the contrast liquid to travel from the start (proximal; FIG. 3, 304) of the vessel segment to the end (distal; FIG. 3, 305) of the vessel segment (contrast bolus propagation time). The length can be determined from the X-ray angiographic image(s), for example by using CAAS QCA3D or CAAS QCA. The time it takes for the contrast liquid to travel through the vessel segment can be determined by counting the number of frames it takes for the contrast liquid to travel from the start (proximal) to end (distal) position within the vessel segment of interest. To convert this count into seconds, the number of frames can be divided by the frame rate of the X-ray acquisition, for instance 15 frames/sec.

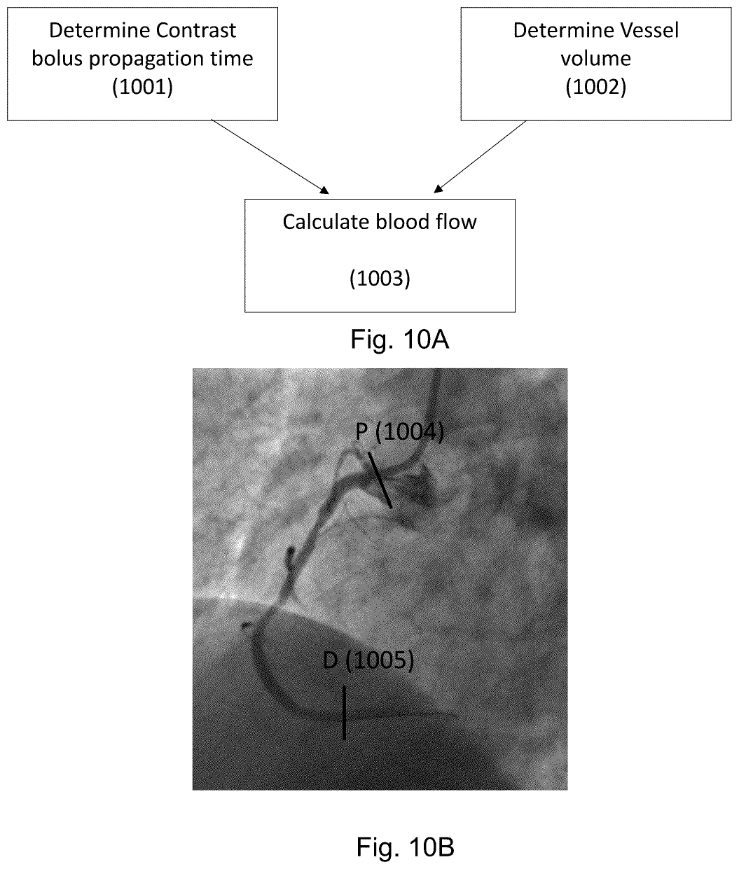

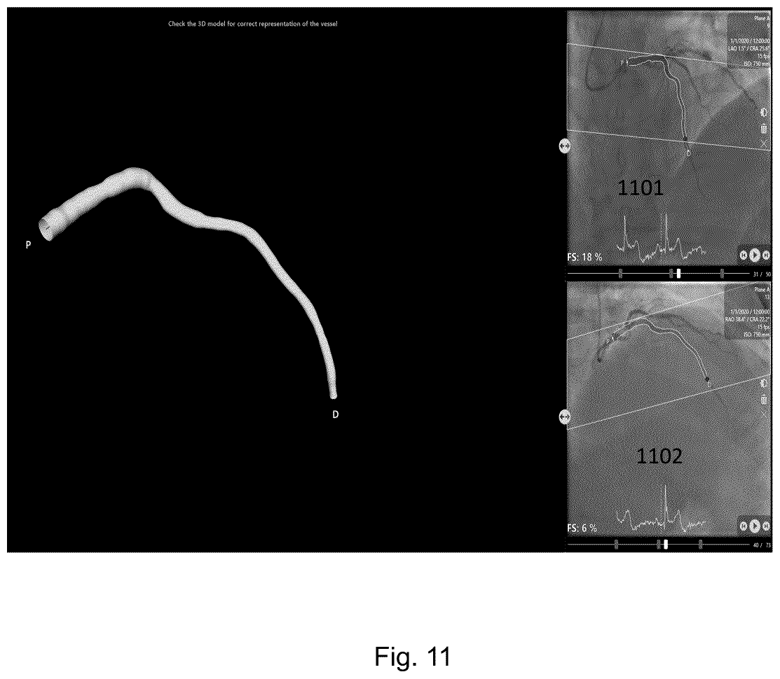

As shown in FIG. 10A, the volumetric flow rate in step 102 can also be derived using the vessel volume (represented by block 1002) divided by the time the contrast bolus travels from proximal location (1004) to its distal location (1005) within the X-ray angiographic image sequence as shown in the example image frame of FIG. 10B. We call this the contrast bolus propagation time (in seconds) represented by block 1001 of FIG. 10A. Both the vessel volume (1002) and contrast bolus propagation time (1001) must be determined over the same vessel segment, i.e., between the proximal position (1004) and distal position (1005). The vessel volume (1002) can be determined based on a 3D reconstruction (1103) of the vessel using two or more X-ray angiographic projections with different viewing angles (1101) and (1102), for example using CAAS QCA3D as shown in FIG. 11. Alternatively, the vessel volume can be determined from a single two-dimensional X-ray angiographic image. In case of a single two-dimensional X-ray projection, the vessel area can be calculated using the diameter along the segmented artery (between 1004 and 1005) and be converted into an area by assuming circularity of the vessel, or by using densitometry in which the cross-sectional area is related to the grey values representing the X-ray absorption, for example by using CAAS QCA (Gronenschild et al, “CAAS. II: A second generation system for off-line and on-line quantitative coronary angiography”, catheterization and cardiovascular diagnosis vol. 33,1 (1994): 61-75). By integrating the cross-sectional areas over the path of the vessel (between 1004 and 1005), the vessel volume can be calculated. Alternatively, the contrast bolus propagation time can be determined by counting the frames it takes for the contrast dye to travel from a proximal position to a distal position divided by the frame rate of the X-ray angiographic image sequence 101.

The contrast propagation time (1001, from FIG. 10A) can also be determined by converting the determined contrast bolus velocity (301 from FIG. 3) into time by dividing the vessel length by the determined contrast bolus velocity (301) resulting in a contrast propagation time. In case the velocity is determined in a two-dimensional image, the length can be derived from this two-dimensional image, for example be performed by using CAAS QCA, or obtained by QCA3D. In case the velocity is determined using 3D (as described before), the length should also be determined in 3D, for example by using CAAS QCA3D. By dividing the vessel volume 1002, as determined before, by this contrast propagation time 1001 the volumetric flow rate 102, or 1003 can be determined. This method is especially of interest in case the contrast bolus velocity (301) is determined using two-dimensional image information as in this situation the determined velocity can be incorrect due to foreshortening effects present in the two-dimensional X-ray angiographic image. For instance, when the contrast bolus velocity (301) is determined by the automatic bolus tracking algorithm using two-dimensional image data, as described before in this patent application. The foreshortening effects can be removed by converting the contrast bolus velocity (301) into a contrast bolus propagation time (1001) by dividing the vessel length, based on the two-dimensional image data, by the determined contrast bolus velocity (301). Using this contrast bolus propagation time (1001) the coronary blood flow can be calculated using the vessel volume (1002) based on 3D information for example by using CAAS QCA3D (as illustrated in FIG. 11 and represented by 1002). Alternatively, the vessel volume can be determined from a single two-dimensional X-ray angiographic image. In case of a single two-dimensional X-ray projection, the vessel area can be calculated using the diameter along the segmented artery and be converted into an area by assuming circularity of the vessel, or by using densitometry in which the cross-sectional area is related to the grey values representing the X-ray absorption, for example by using CAAS QCA (Gronenschild et al, “CAAS. II: A second generation system for off-line and on-line quantitative coronary angiography”, catheterization and cardiovascular diagnosis vol. 33,1 (1994): 61-75). By integrating the cross-sectional areas over the length of the vessel, the vessel volume can be calculated.

Contrast liquid has a higher viscosity (n) compared to blood and saline (saline is used for instance during invasive IMR measurements), this viscosity difference can be taken into account to correct the contrast flow into a blood or saline flow. Assume the human body tries to keep the pressure difference constant, Hagen-Poiseuille (equation 2) teaches that the velocity must decrease in case the vessel length (L) and radius (r) do not change.

$$Δ P = \frac{8 * η * v * L}{{r}^{2}} Δ P = \frac{8 * η * v * L}{{r}^{2}} \qquad Eqn . ( 2 )$$

ΔP=pressure drop along the coronary artery
η=blood viscosity
v=coronary velocity
L=coronary vessel length
r=radius of the coronary artery

When using the assumption that the pressure drop must remain constant, the multiplication of η*v within equation 2 must be constant for all fluids resulting in the following correction to calculate the blood velocity from contrast velocity (equation 4).

$${η}_{b l o o d} {v}_{b l o o d} = {η}_{contrast} * {v}_{contrast} \qquad Eqn . ( 3 )$$

$${v}_{b l o o d} = \frac{{η}_{c o n t r a s t}}{{η}_{b l o o d}} * {v}_{contrast} \qquad Eqn . ( 4 )$$

ΔP=pressure drop along the coronary artery
η_{blood}=blood viscosity
ηcontrast=viscosity of the contrast liquid
v_{blood}=coronary velocity
v_{contrast}=velocity of the contrast liquid

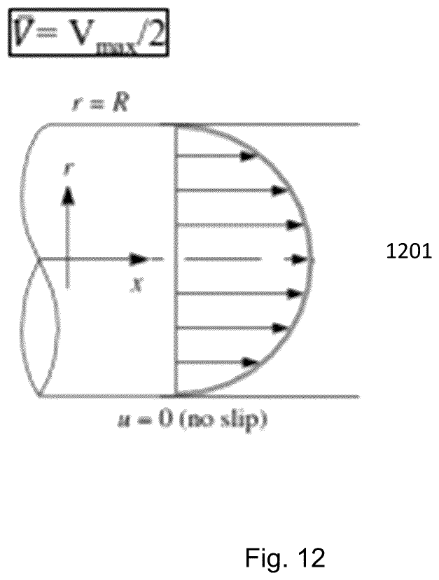

When determining the contrast bolus velocity or contrast bolus propagation time it is important to consider if the average velocity (or propagation time) of all particles is measured or the fastest particles. When measuring the fastest particles, a correction might be needed to convert the contrast bolus propagation time or contrast bolus velocity of these fastest particles into the average propagation time or velocity of all particles. An example of that correction is assuming a Poiseuille profile having the property that the average velocity is half of the maximum velocity within the parabolic velocity profile (1201) as shown in FIG. 12.

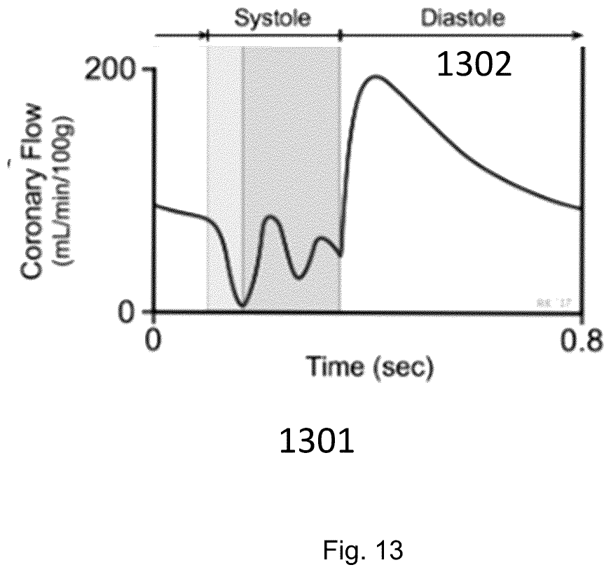

During one cardiac cycle the coronary volumetric flow rate and coronary flow velocity are not constant. During contraction of the myocardium (systole) the coronary flow is low and during relaxation of the myocardium muscle (diastole) the flow is high as illustrated by FIG. 13 (picture 1301). When the coronary volumetric flow rate and coronary flow velocity is determined within a sub part of the cardiac cycle, a correction can be applied to correct the calculated coronary volumetric flow rate or coronary flow velocity to the average coronary volumetric flow rate or average coronary flow velocity over an entire cardiac cycle. This can be done as explained in U.S. patent application Ser. No. 15/971,275 “Method and Apparatus for determining blood velocity in X-ray angiographic images” in which v_{local} being the velocity computed within a part of the cardiac cycle, is corrected to an average velocity vₘₑₐₙ using a generic full velocity cycle profile (f(x)). A correction factor is calculated being the area under the curve (integral) of the full generic velocity profile (f(x)) divided by the integral of f(x) over the part of the heart cycle covered within the v_{local} computation. Using this correction factor the v_{local} Can be corrected to vₘₑₐₙ. The same correction factor can be used in case coronary flow is determined within a part of the heart cycle.

Alternatively, such a correction to correct the calculated coronary volumetric flow rate or coronary flow velocity (v_{local}) to the average coronary volumetric flow rate or average coronary flow velocity (vₘₑₐₙ) over an entire cardiac cycle can be based on the ECG signal extracted from the DICOM file. Alternatively, this ECG signal can be extracted from the image sequence as for instance disclosed by U.S. Pat. No. 11,707,242 “Method and system for dynamic coronary roadmapping”. Another method to perform such correction can be based on the coronary motion observed within the X-ray angiographic sequence. This coronary motion can for instance be extracted from the image sequence as disclosed by U.S. Pat. No. 11,707,242 “Method and system for dynamic coronary roadmapping.”

The above corrections can be extracted by correlation of the calculated coronary volumetric flow rate or coronary flow velocity (v_{local}) to the true coronary volumetric flow rate or the average coronary flow velocity (vₘₑₐₙ) based on invasive measurements. Once the correction is determined, the coronary flow velocity (v_{local}) can be adjusted based on the ECG signal or the average coronary motion (vₘₑₐₙ) can be calculated.

The flow through the myocardium can be regulated based on the blood supply it needs by adjusting its resistance. The coronary blood flow should be reproducible, and the coronary blood flow should be measured in the same state (amount of stress) of the patient. During the diastolic wave-free period (1302) of the cardiac cycle the microvascular resistance is naturally minimized without the need of hyperemia induced by the administration of a vasodilator (Sen, Sayan et al. “Development and validation of a new adenosine-independent index of stenosis severity from coronary wave-intensity analysis: results of the ADVISE (ADenosine Vasodilator Independent Stenosis Evaluation) study”, Journal of the American College of Cardiology vol. 59,15 (2012): 1392-402). This means that the flow must be similar between the wave-free period (1908) and during pharmacological vasodilation and therefore, the wave-free period can be used to determine the flow using angiographic images acquired in resting conditions having the same magnitude and variability. This wave-free period is a period within the diastolic phase of the cardiac cycle. The coronary flow within the current patent application is determined by evaluating the contrast liquid propagation through the coronary artery. To make sure the coronary flow is measured during this period, the contrast liquid injection must be triggered based on an electrocardiogram ensuring that the contrast liquid travels through the coronary artery of interest in this diastolic wave-free period. Possible delays between the start of contrast injection and passing of contrast liquid in the vessel of interest may be taken into account. This can be determined in a patient population or estimated based on normal coronary blood flow or velocity and distance the contrast liquid must travel. Alternatively, the coronary velocity is derived in accordance with the methods as described with reference to FIG. 3, but limited to the wave-free period. This can be accomplished by fitting a line (402) only through the (time) points within the wave-free period. Further the calculation of the coronary blood flow (303) in accordance with equation 1. The cross-sectional areas used in equation 1 are limited to the vessel path in which the contrast bolus travels within the wave-free period. The same is true in case the coronary flow is derived in accordance with the methods as described with reference to FIG. 10A, the determination of the contrast bolus propagation time (1001) needs to be limited to the wave-free period, and the vessel volume (1002) needs to be limited to the vessel path corresponding to the path the bolus front travels within the wave-free period. The frame within the image sequence in which the wave-free period is applicable can be determined by the ECG signal corresponding the image data (101), for instance as part of the DICOM file. In case the ECG signal is not available, the cardiac cycle information can be extracted as disclosed by U.S. patent application Ser. No. 16/739.718 “Method and system for dynamic coronary roadmapping”.

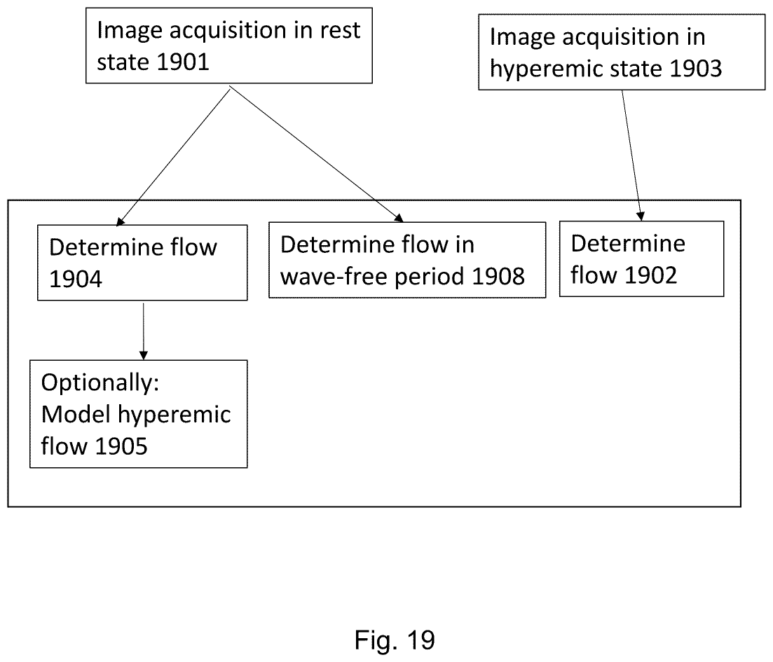

Contrast liquid, to some extent, induces hyperemia (Tatineni, S et al. “The effects of ionic and non-ionic radiographic contrast media on coronary hyperemia in patients during coronary angiography”, American heart journal vol. 123,3 (1992): 621-7). When acquiring X-ray angiographic images of the patient while the patient is in resting state (FIG. 19, 1901), a hyperemic effect will be present in the flow determination induced by the contrast liquid used to image the coronary arteries using X-ray. This hyperemia effect reduces the variability of flow measurements.

In case the flow is needed in another state of the patient (e.g., rest versus hyperemia) compared to the state in which the X-ray image data is acquired, it is optionally possible to correct the determined flow (1904) for example from rest (1901) into hyperemia (1905) as shown in FIG. 19. This can be done by modeling (for example polynomial fit, machine learning etc.) based on clinical data, e.g., invasive measurements of rest and hyperemic flow and X-ray image data, or can be multiplication with a constant factor, e.g., 2.5 (Johnson, Nils P et al. “Does the instantaneous wave-free ratio approximate the fractional flow reserve?”, Journal of the American College of Cardiology vol. 61,13 (2013): 1428-35), or functions like, explained in Tu, Shengxian et al. “Diagnostic Accuracy of Fast Computational Approaches to Derive Fractional Flow Reserve From Diagnostic Coronary Angiography: The International Multicenter FAVOR Pilot Study”, JACC. Cardiovascular interventions vol. 9,19 (2016): 2024-2035. Alternatively, an X-ray acquisition of the patient during hyperemic (1903) can be used to determine the hyperemic flow (1902). The modeling described in this section allows for computation of microvascular resistance within different states of the patient (e.g., rest versus hyperemia).

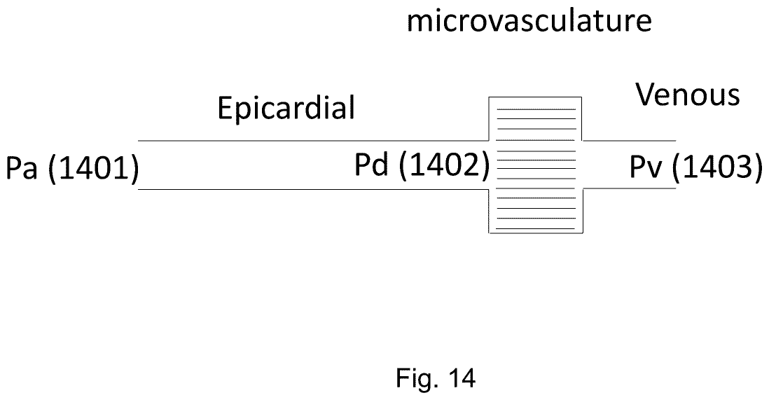

In step 103 of FIG. 1, the pressure drop is determined. The pressure drop (4P) is the difference between the pressure at distal position within the coronary artery (1402) supplying the myocardium with blood (P_{d}) and the venous pressure (Pᵥ) (1403) as shown in FIG. 14. The distal coronary artery pressure can be determined non-invasively from X-ray angiography using geometrical parameters and optionally patient specific data like aortic pressure. An example is the usage of vFFR workflow within CAAS Workstation 8.5 (Pic Medical Imaging, the Netherlands) as described by Masdjedi et al., “Validation of 3-Dimensional Quantitative Coronary Angiography based software to calculate Fractional Flow Reserve: Fast Assessment of STenosis severity (FAST)-study”, EuroIntervention: journal of EuroPCR in collaboration with the Working Group on Interventional Cardiology of the European Society of Cardiology 2019. Other methods to determine the distal coronary pressure includes the method as described in U.S. patent application Ser. No. No. 15/551,162 entitled “Method and apparatus for quantitative flow analysis” which discloses a method to quantify pressure loss based on a 3D reconstruction of the coronary tree using X-ray angiography in combination with a multi-scale functional model which incorporates a 1D/0D model, or methods like explained by Kirkecide et al. “Assessment of coronary stenoses by myocardial perfusion imaging during pharmacologic coronary vasodilation. VII. Validation of coronary flow reserve as a single integrated functional measure of stenosis severity reflecting all its geometric dimensions”, Journal of the American College of Cardiology vol. 7,1 (1986): 103-13, and Gould et al. “Experimental validation of quantitative coronary arteriography for determining pressure-flow characteristics of coronary stenosis”, Circulation vol. 66,5 (1982): 930-7. The venous pressure can be measured invasively for example by a central venous catheter in the venae cavae or non-invasively for example as explained by Thalhammer, Christoph et al. “Noninvasive central venous pressure measurement by controlled compression sonography at the forearm”, Journal of the American College of Cardiology vol. 50,16 (2007): 1584-9. For the venous pressure it is also possible not to perform a measurement but assume a fixed value, for example 10 mmHg or even neglect the venous pressure.

Optionally the distal pressure can be determined invasively using a pressure wire inside the coronary artery.

In step 104 of FIG. 1, an index for microvascular dysfunction or resistance is calculated. In embodiments, the index is quantitative data that represents the amount of dysfunction or resistance in the microvascular tissue of the myocardium that is supplied with blood through the coronary artery under investigation. An example of such an index is the microvascular resistance calculated by the fraction of the pressure drop of step 103 from FIG. 1 and the volumetric flow rate determined in step 102 from FIG. 1:

$$R = \frac{dP}{Q} \qquad Eqn . ( 5 )$$

R=Microvascular resistance
dP=pressure drop determined in step 103
Q=coronary volumetric flow rate determined in step 102

An advantage of an index as given in equation 5 compared to the existing index of microvascular resistance (IMR) determined from X-ray angiography (De Maria et al. “Angiography-derived index of microcirculatory resistance as a novel, pressure-wire-free tool to assess coronary microcirculation in ST elevation myocardial infarction”, the international journal of cardiovascular imaging vol. 36,8 (2020): 1395-1406 and Scarsini et al. “Angiography-derived index of microcirculatory resistance (IMRangio) as a novel pressure-wire-free tool to assess coronary microvascular dysfunction in acute coronary syndromes and stable coronary artery disease”, the international journal of cardiovascular imaging vol. 37,6 (2021): 1801-1813, and Fernández-Peregrina et al. “Angiography-derived versus invasively-determined index of microcirculatory resistance in the assessment of coronary microcirculation: A systematic review and meta-analysis”, catheterization and cardiovascular interventions: official journal of the Society for Cardiac Angiography & Interventions vol. 99,7 (2022): 2018-2025) is that the approach as presented in current patent application depends on the coronary volumetric flow rate (Q) instead of contrast propagation time (T). Methods based on the contrast propagation time as described by above references (De Maria et al, Scarsini et al, and Fernández-Peregrina et al), assume that coronary vessel diameter is constant between patients in order to compare the index of microvascular resistance between patients. However, coronary vessel diameter varies between patients (Dodge et al. “Lumen diameter of normal human coronary arteries. Influence of age, sex, anatomic variation, and left ventricular hypertrophy or dilation”, circulation vol. 86,1 (1992): 232-46), resulting for example in different propagation times, due to difference in vessel diameter, with the same flow. Even the use of coronary velocity instead of contrast propagation time would not solve the problem of coronary diameter differences, because it does not take the cross-sectional area into account, only coronary volumetric flow rate incorporates the cross-sectional area. Also, in case of a stenosis, the decrease in diameter at the stenosis results in a higher coronary velocity decreasing the propagation time while the coronary volumetric flow rate is not increased. The coronary volumetric flow rate (Q) takes the cross-sectional area of the coronary artery that supplies blood to the microvascular tissue of the myocardium into account, which can correct for this higher velocity and lower propagation time at a stenosis or in smaller vessels. This last aspect makes the proposed approach suitable for determination of the absolute myocardium resistance for an indication of microvascular dysfunction (in the presence of obstructive coronary disease) by removing the variability induced by diameter and cross-sectional area differences between patients.

Optionally, in step 105, the index for microvascular dysfunction or resistance can be normalized to correct for differences between patients in for example amount of myocardial blood supply and microvascular resistance. This normalization can be based on:
cardiac mass
coronary volume
coronary artery cross-sectional area
patient weight, height, body surface area (BSA) or body mass index (BMI)
heart “dominance”
combinations: e.g., vessel-specific cardiac mass (combination of cardiac mass and heart dominance)

### Normalization Based on Cardiac Mass

The myocardium mass or myocardium volume (cardiac mass, cardiac volume) influences the blood supply needed. The bigger the heart muscle the more blood supply needed. This blood supply is regulated by the microvascular resistance and therefore there will be a correlation of the cardiac mass or volume with microvascular resistance.

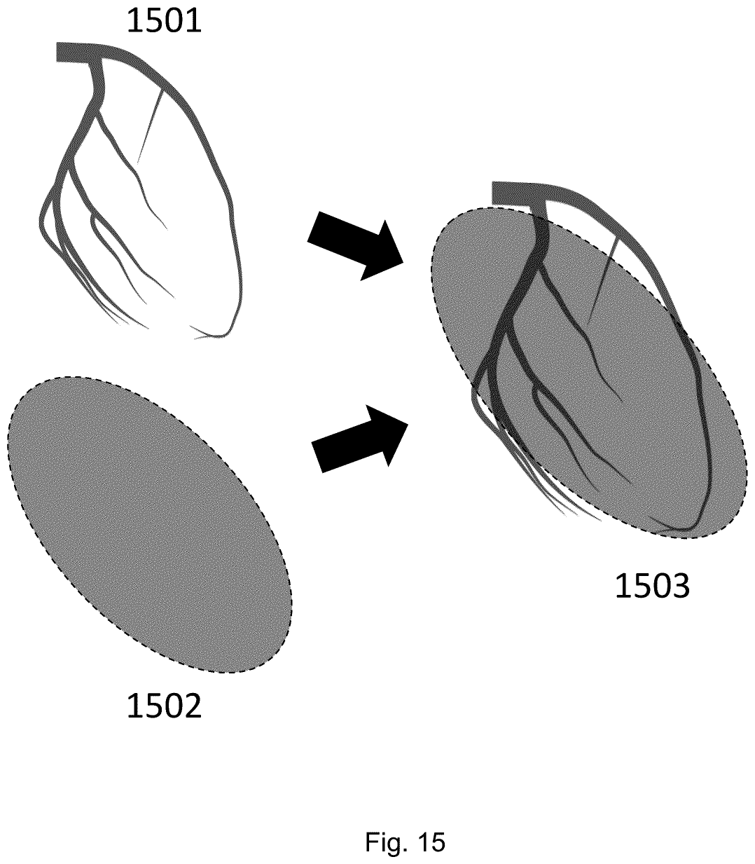

The myocardial mass or volume can be determined from X-ray angiography. One method is to segment the coronary arteries or the coronary tree (1501) in one or multiple projections providing information about the shape/geometry/size of the myocardium as shown in FIG. 15. The myocardium shape (1502) can be fitted through the shape of the coronary arteries (1503). In case multiple projections are used, a 3D reconstruction of the coronaries/coronary tree can be made to determined and used to model the myocardial shape more accurately and optionally in 3D.

Optionally, in case a single projection is used, the visible myocardium shape differs between projection angles. The shape determined within a single projection can be corrected using prior knowledge of myocardium shape based on patient populations and the projection angles of the X-ray angiographic image.

Optionally, in case one coronary artery is visible within the X-ray angiographic image, for example the right coronary artery, the shape of the entire myocardium can be estimated using prior knowledge of myocardium shape based on patient populations in combination with heart dominance (Left Dominant, etc.).

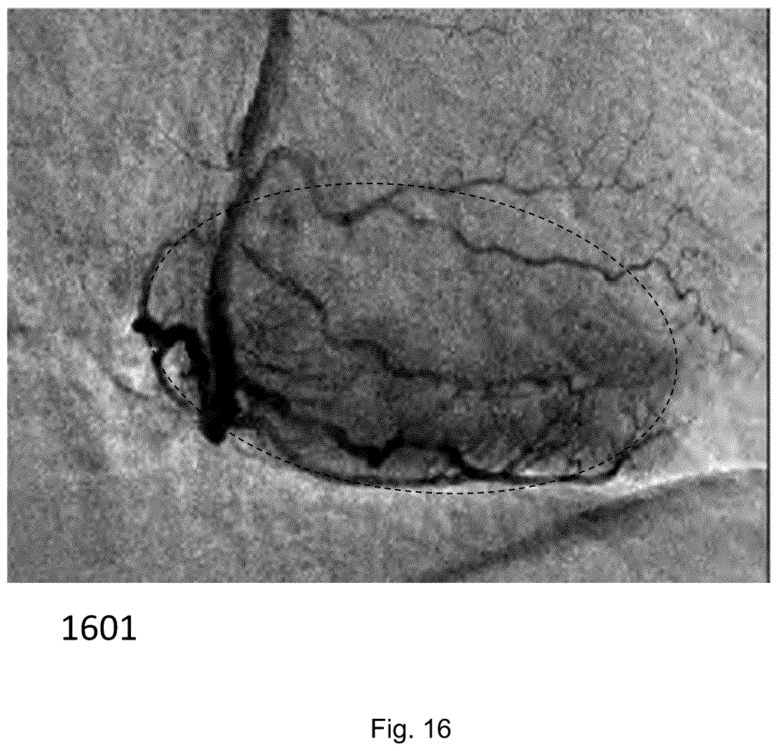

Another method to determine the myocardial mass is by delineating the myocardial blush effect on the X-ray angiography, as illustrated in 1601 of FIG. 16. One can also, instead of delineation, fit a predefined shape through the myocardium, e.g., an elliptical shape. In case multiple projections are used, delineation or shape fitting from these multiple projection angles can be used to determine the myocardial shape and mass more accurately and optionally in 3D.

Optionally, this delineated myocardium within a single X-ray angiographic projection can be made more accurate based on the projection angles of the X-ray angiographic image and prior knowledge of the myocardium shape based on patient populations.

Another method to perform the myocardial mass estimation is using coronary computed tomography angiography (CCTA) image data. From CCTA image data the myocardium is visible and can be segmented. Also, segmentation of the coronary arteries from CCTA image data can be used to determine or guide or estimate the myocardial mass.

According to Choy, Jenny Susana, and Ghassan S Kassab, “Scaling of myocardial mass to flow and morphometry of coronary arteries”, journal of applied physiology (Bethesda, Md. : 1985) vol. 104,5 (2008): 1281-6 there is an exponential relation between coronary volumetric flow rate (Q_{moddel}) and the myocardial mass (M_{myo}) given by equation 6. The parameters Q₀ and b from equation 6 can be obtained from Choy et al. or fitted on coronary volumetric flow rate data and cardiac mass data of patient populations. Such a relation (equation 6) can be used to scale the patient specific volumetric flow rate (Q) as determined in step 102 to a volumetric flow rate belonging to a reference cardiac mass. First a scaling factor (S) will be calculated as the ratio between the modeled volumetric flow rate (Q_{myo}) from equation 6, using the patient specific cardiac mass and the reference modeled volumetric flow rate (Q_{ref}) belonging to a reference cardiac mass (M_{ref}) also calculated, see equation 7, for example the average myocardium mass of a patient population can be used as reference cardiac mass. This scaling factor can then be used to normalize the patient specific volumetric flow rate as determined in step 102 towards a flow belonging to a reference cardiac mass (Q_{normalized}), see equation 8. The normalized microvascular resistance can then be calculated using the normalized flow as illustrated in equation 9.

$${Q}_{m o ddel} = {Q}_{0} * {M}_{m y o}^{b} \qquad Eqn . ( 6 )$$

$$S = \frac{{Q}_{myo}}{{Q}_{ref}} = \frac{{Q}_{0} * {M}_{m y o}^{b}}{{Q}_{0} * {M}_{ref}^{b}} \qquad Eqn . ( 7 )$$

$${Q}_{normalized} = \frac{Q}{S} \qquad Eqn . ( 8 )$$

$${R}_{normalized} = \frac{d P}{{Q}_{n o r m a l i z e d}} \qquad Eqn . ( 9 )$$

Q_{moddel}=coronary volumetric flow rate modeled using Choy et al.
Q=coronary volumetric flow rate determined in step 102
Q₀=normalization constant
Q_{ref}=reference volumetric flow rate belonging to a reference cardiac mass
Q_{normalized}=patient specific volumetric flow rate normalized to a reference cardiac mass
S=Scaling factor
M_{myo}=determined patient specific myocardium mass
M_{ref}=reference myocardium mass
b=power-law exponent
R_{normalized}=normalized microvascular resistance
Alternatively, normalization can be done by dividing the resistance by the cardiac mass or volume to obtain a resistance per mass or volume unit, see for example equation 10.

$${R}_{n o r m a l i z e d} = \frac{R}{M} = \frac{\frac{d P}{Q}}{M} [ \frac{\frac{mm Hg}{m 3}}{Kg} ] \qquad Eqn . ( 10 )$$

R_{normalized}=microvascular resistance per unit of cardiac mass
R=microvascular resistance determined in step 104 (or a normalized version)
M=myocardium mass
Q=volumetric flow rate determined in step 102
dP=pressure drop determined in step 103

Alternatively, the relationship between cardiac mass and myocardial resistance (R_{ref} as function of M_{myo}) can be determined in a healthy population. This relationship can be used to determine the resistance relative to the healthy resistance belonging to the patient specific cardiac mass. A relative resistance of 1.0 means a healthy myocardium. The more the value above 1.0 means the more microvascular disease.

$${R}_{rel} = \frac{R}{{R}_{ref} ( {M}_{myo} )} \qquad Eqn . ( 11 )$$

R=microvascular resistance as determined in step 104
M_{yo}=myocardial mass
Rᵣₑₗ=microvascular resistance relative to healthy
R_{ref}(M_{myo})=healthy microvascular resistance as function of myocardial mass

This relative resistance is relative to a healthy resistance, it can also be scaled between a healthy and (near) death tissue resistance. This results in a scaled resistance between 0 and 1. Resistance is minimal (Rₘᵢₙ) 0 when the microvascular tissue is healthy and 1 in case the resistance is maximal (Rₘₐₓ) representing maximum amount of diseased tissue in a patient. This maximum resistance can be determined using equation 12 which depends on the pressure drop determined in step 103 and a minimum minimal volumetric flow rate (Qₘᵢₙ) needed for the myocardium to stay alive. The relative resistance (Rᵣₑₗ) can then be calculated using equation 13.

$${R}_{max} = \frac{d P}{{Q}_{min}} \qquad Eqn . ( 12 )$$

$${R}_{rel} = \frac{R - {R}_{min} ( {M}_{myo} )}{{R}_{max} - {R}_{min} ( {M}_{myo} )} \qquad Eqn . ( 13 )$$

Rₘₐₓ=maximum microvascular resistance at which the patient is still alive
Qₘᵢₙ=minimal volumetric flow rate at which the myocard stays alive
R=microvascular resistance as determined in step 104
dP=pressure drop determined in step 103
Rᵣₑₗ=microvascular resistance scaled between healthy (0) and death (1) tissue
Rₘᵢₙ(M_{myo})=healthy microvascular resistance as function of myocardial mass

### Normalization Based on Coronary Volume

In this normalization approach we assume that with a larger heart (compared to average), have a higher myocardium volume resulting in a higher myocardium blood supply. Therefore, the coronary (tree) volume can be used to normalize the volumetric flow rate (Q_{normalized}). This can be done by calculating a scaling factor (S) between the measured patient specific volume (V_{measured}) and a reference coronary volume (Vᵣₑₛ), see equation 14, and multiply the patient specific coronary flow with this scaling factor to obtain the normalized coronary volumetric flow rate (Q_{normalized}), equation 15, which can be used in equation 16 to obtain the microvascular resistance.

$$S = \frac{{V}_{ref}}{{V}_{m e a sured}} \qquad Eqn . ( 14 )$$

$${Q}_{n o r m a l i z e d} = S * Q \qquad Eqn . ( 15 )$$

$${R}_{n o r m a l i z e d} = \frac{dP}{{Q}_{n o r m a l i z e d}} \qquad Eqn . ( 16 )$$

S=Scaling factor to normalized flow based on coronary (tree) volume
V_{measured}=measured patient specific coronary (tree) volume
V_{ref}=reference coronary (tree) volume
Q=coronary volumetric flow rate determined in step 102
Q_{normalized}=normalized coronary volumetric flow rate
R_{normalized}=normalized microvascular resistance
dP=pressure drop determined in step 103

The scaling factor of equations 14 and 15 can also be based on other relationships between the reference and measured volume, for example exponential, logarithmic, etc.

Optionally a relationship (equation/model) between the coronary volume and flow can be determined based on patient population(s). This relationship can be used to normalize the determined patient specific flow towards a flow belonging to a reference patient, e.g., a patient with a predefined coronary volume.

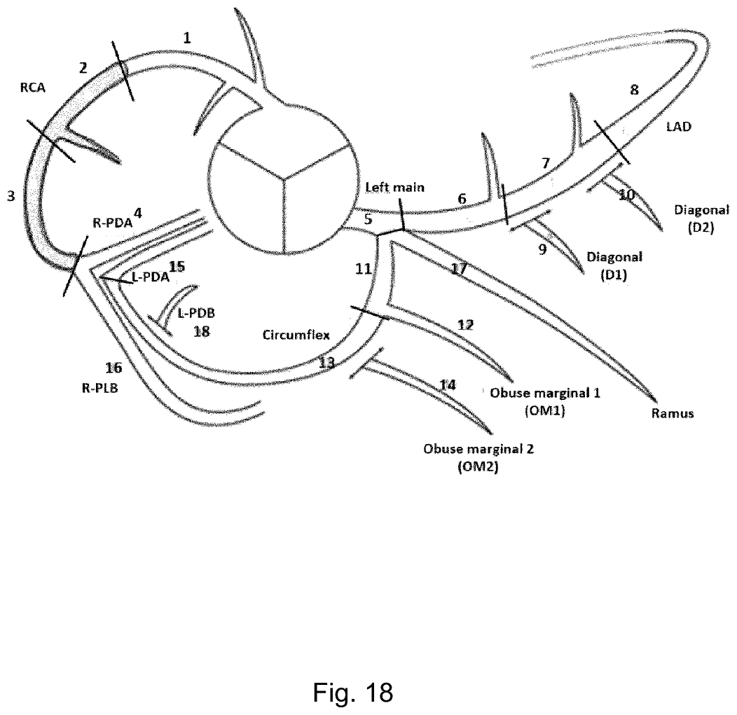

When using coronary volume for normalization, the section of the coronary tree over which the volume must be calculated must be defined/specified to make sure the same section is taken within all patients. This section can be defined using, for example definitions according to a general model for the coronary tree according to the American Heart Association, as illustrated in FIG. 18.

### Normalization Based on Coronary Cross-Sectional Area

Similar to the coronary volume we can use the cross-sectional area of the coronary artery at a specified location, e.g., left main, ostium, or at a specific branch or bifurcation within the coronary tree. Like the volume method a scaling factor (S) can be calculated using a reference cross-sectional area (A_{ref}) and the measured cross-sectional area (A_{measured}) at the specified location to calculate a normalized coronary volumetric flow rate (Q_{normalized}). This normalized coronary volumetric flow rate can be used to calculate the microvascular resistance using equation 19.

$$S = \frac{{A}_{ref}}{{A}_{m e a s u r e d}} \qquad Eqn . ( 17 )$$

$${Q}_{normaiized} = S * Q \qquad Eqn . ( 18 )$$

$${R}_{n o r m a l i z e d} = \frac{dP}{{Q}_{n o r m a l i z e d}} \qquad Eqn . ( 19 )$$

S=Scaling factor to normalized flow based on cross-sectional area
A_{measured}=measured patient specific coronary cross-sectional area
A_{ref}=reference coronary cross-sectional area
Q=coronary volumetric flow rate determined in step 102
Q_{normalized}=normalized coronary volumetric flow rate
R_{normalized}=normalized microvascular resistance
dP=pressure drop determined in step 103

The scaling factor of equations 17 and 18 can also be other relationships between the reference and measured cross-sectional area, for example exponential, logarithmic, etc.

Also using the cross-sectional area, optionally a relationship (equation/model) between the cross-sectional area at the specified location and the flow can be determined based on data from a population to normalize the flow towards a flow belonging to a reference patient, e.g., a patient with a predefined cross-sectional area at the specified location.

### Normalization Based on Patient Weight, Length, BSA or BMI

Larger/bigger/longer people will have a larger myocardium to provide the entire body with blood. Using parameters like patient weight, length, BSA or BMI the cardiac mass can be modelled (e.g., M_{myo}(weight), M_{myo}(length), M_{myo}(BSA) or M_{myo}(BMI)). These models can, for example, be linear or quadratic relationships between the parameter and cardiac mass. These models can be derived from a (patient) population.

### Normalization Based on Heart Dominance

In case the blood flow is determined using one of the three mayor coronary arteries, i.e., left anterior descending (LAD), left circumflex (LCX), right coronary artery (RCA) the relative amount of the total myocardium supplied by this coronary depends on the heart dominance (i.e., left dominant, right dominant, codominant). This should be considered to be able to compare measurements of vessels of different heart dominance. To do this the flow determined from the X-ray angiography step 102 must be corrected. For example, suppose a left dominant heart provides a certain percentage of the myocardial mass with blood called blood supply fraction (BSF), the coronary volumetric flow rate should be corrected to 100%, see equation 20. Using this corrected coronary volumetric flow rate, the microvascular resistance can be calculated using equation 21.

$${Q}_{100 \%} = \frac{Q}{BSF} * 100 \qquad Eqn . ( 20 )$$

$${R}_{n o r m a l i z e d} = \frac{dP}{{Q}_{100 \%}} \qquad Eqn . ( 21 )$$

Q₁₀₀%=coronary volumetric flow rate corrected for total myocardium
Q=coronary volumetric flow rate determined in step 102
BSF=blood supply factor
R_{normalized}=normalized microvascular resistance
dP=pressure drop determined in step 103

### Combination of Normalization Methods

A combination of the above-mentioned normalization methods can be

used. For example, the mass of the part of the myocardium supplied by a specific vessel (e.g., LAD, LCX, RCA) can be determined using heart dominance. This can be used to normalize a resistance.

### Reduce Variability

Because the flow and pressure vary along the coronary arteries, variability can be decreased by determining the flow and distal pressure of the coronary artery at a specified location within the coronary tree, for example a specified amount of centimeters after a specific bifurcation.

### Alternative Approaches

### Image Derived Coronary Blood Flow as an Index for Microvascular Dysfunction

Microvascular resistance is derived from the coronary blood flow and pressure as described before. The main demand of a properly functioning myocardium is sufficient supply of oxygen by the coronary arteries and microvasculature. The coronary blood flow is a quantity that is directly related to the amount of oxygen transported to the myocardium. Therefore, the calculated coronary blood flow (step 102 of FIG. 1) can also be used as an index to indicate microvascular dysfunction or predict future events such as cardiovascular death, myocardial infarction, hospitalization for heart failure, or ischemia-driven revascularization. This method is particularly of interest in situation in which the pressure drop of the coronary artery of interest is negligible or cannot be calculated due to missing information of patient specific information required for accurate calculation of the pressure drop, for instance the aortic pressure at rest when using vFFR. The coronary blood blow can optionally be normalized in a similar way as described in step 105 of FIG. 1, to be able to compare the flow between patients. On order to distinguish between healthy coronary blood flow and reduced coronary blood flow for instance due to microvascular dysfunction a coronary blood flow threshold can used. Several options can be followed to define this coronary blood flow threshold. For instance, based on the statistical difference in coronary blood flow in a healthy population and a disease population. The disease population can be identified by invasive IMR or invasive bolus thermodilution. Another approach to define the disease population is identifying an event after a predefined follow up period, for instance one year. The event can be defined as major adverse cardiovascular events, a composite of cardiovascular death, myocardial infarction, hospitalization for heart failure, or ischemia driven revascularization. Having the coronary blood flow as derived by the methods described by this patent application, the statistics involved to define the threshold can be lowest, middle, or highest tertile of the derived coronary blood flow within the whole population (healthy and disease), or other statistical test to distinguish two groups. The above-described approach to distinguish between healthy coronary blood flow and reduced coronary blood flow can also be applied to the calculated microvascular resistance as described by this patent application.

### Improve Current Established IMR Measurements

Quantitative X-ray image data analysis can improve the established IMR method to assess microvascular disease (Fearon et al. “Novel index for invasively assessing the coronary microcirculation”, circulation vol. 107,25 (2003): 3129-32). IMR calculated by equation 22 is based on the assumption that the vascular volume is constant. As described before there is a variety of vascular volume between patients. To overcome this assumption the IMR index can be corrected for differences in vascular volume between patients by assessing this volume using image analysis, for example using CAAS QCA or CAAS QCA3D.

IMR=P_{d}*Tₘₙ

IMR=P_{d}*Tₘₙ Eqn. (22)
IMR=Index of Microvascular Resistance myocardium
P_{d}=blood pressure distal in coronary artery
Tₘₙ=Mean transit time

To incorporate the vessel volume, equation 22 can substituted by equation 23 in which V is the vascular volume.

$$IMR = {P}_{d} * \frac{{T}_{m n}}{V} IMR = {P}_{d} * \frac{{T}_{m n}}{V} \qquad Eqn . ( 23 )$$

IMR=Index of Microvascular Resistance myocardium
P_{d}=blood pressure distal in coronary artery
Tₘₙ=Mean transit time
V=coronary vascular volume

Alternatively, in case of a stenosis the mean transit time (Tₘₙ) will be underestimated due to increased coronary velocity in the smaller cross-sectional area at the stenosis. The coronary velocity within a coronary artery having a stenosis (Vₛₜₑₙₒₛᵢₛ) can be estimated based on X-ray angiographic image data and the velocity after treatment (Vₜᵣₑₐₜ), i.e., removal of the stenosis, can be predicted, both as explained in U.S. patent application Ser. No. 16/438,955 “Method and Apparatus for quantitative hemodynamic flow analysis”. Using the calculated velocity before and after treatment, the mean transit time determined in the established IMR measurements (Tₘₙ) can be corrected for stenosis error effects as given in equation 24.

$${T}_{mn corrected} = {T}_{m n} * \frac{{V}_{s t e n o s i s}}{{V}_{t r e a t}} {T}_{mn corrected} = {T}_{m n} * \frac{{V}_{stenosis}}{{V}_{treat}} \qquad Eqn . ( 24 )$$

Tₘₙcorrected=Mean transit time corrected for stenosis effects
Tₘₙ=Mean transit time
Vₛₜₑₙₒₛᵢₛ=coronary blood velocity with stenosis
Vₜᵣₑₐₜ=coronary blood velocity after treatment

Alternatively, the distal pressure measurement within the established IMR measurements can be replaced by non-invasive distal pressure calculations using geometrical features of the vasculature extracted from the X-ray image data. For example, as performed within CAAS vFFR. These calculations can optionally be improved by incorporating the flow determined in step 102.

### Alternative Approach for Contrast Bolus Propagation Time

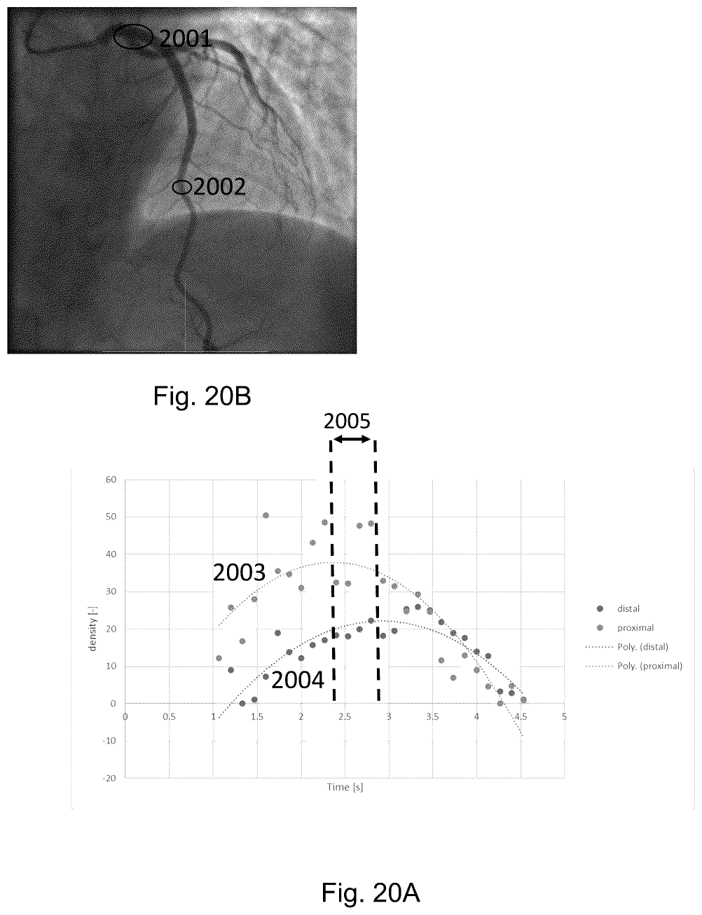

An alternative approach to determine the contrast bolus propagation time as explained in step 102 is by analyzing the density of the contrast at both the proximal and distal location within the coronary artery as shown by FIG. 20A. At a proximal location within the coronary artery, a region of interest (ROI labeled 2001) is indicated as shown in the FIG. 20B. The average, sum, median or some other metric of the pixel values within this ROI, called “density values”, can be determined for all frames of the X-ray angiographic image sequence. These density values can be plotted as a function of time (seconds) using the frame rate of the image acquisition (2003, data points). The same plot can be created for a ROI labeled 2002 at the distal location in the coronary artery, resulting in the graph represented by data points 2004. A curve can be fitted through the density values over time, for example polynomial, for both the proximal and distal ROI (2003 dotted line and 2004 dotted line). By determining the time differences of specific landmarks within the fitted curves, for example the time difference between the peaks of the proximal and distal curve (which is shown as 2005) gives the contrast bolus propagation time. Also, other landmarks can be used, for example center of gravity of the area below the curve, start of upslope and/or downslope, etc. The “density values” as described above can also be the average, sum, median or some other metric of the densitometric pixel values within this ROI. Such densitometric pixel values are obtained by subtracting the background as for instance described in Gronenschild et al, “CAAS. II: A second generation system for off-line and on-line quantitative coronary angiography”, catheterization and cardiovascular diagnosis vol. 33,1 (1994): 61-75. Alternatively, a pre-processing of the image sequences can be performed in order to remove the background layer in the image sequence as for instance disclosed by Hao et al., “Vessel Layer Separation in X-ray Angiograms with Fully Convolutional Network”, Proc. SPIE 10576, Medical Imaging 2018: Image-Guided Procedures, Robotic Interventions, and Modeling or Ma et al., “Layer separation for vessel enhancement in interventional X-ray angiograms using morphological filtering and robust PCA”, Workshop on Augmented Environments for Computer-Assisted Interventions 2017, Springer. pp. 104-113. Optionally, the cardiac motion and breathing motion can be corrected as for instance using the methods as disclosed by U.S. patent application Ser. No. 17/208,373 “Method and system for registering intra-object data with extra-object data”.

### Alternative Approach for Determining an Index for Microvascular Resistance or Dysfunction

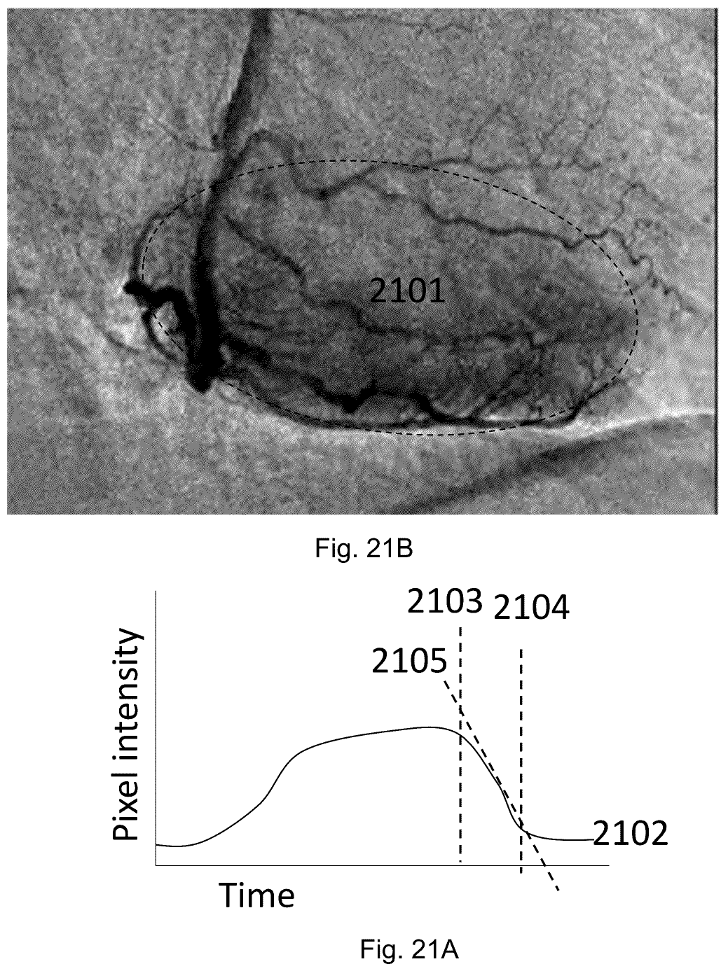

A different approach to evaluate myocardial status without determination of contrast velocity or contrast bolus transit time is by evaluating the contrast density over time within a region of interest (ROI) as illustrated in FIG. 21A. In case information about the global myocardial status (entire myocardium) is needed, an ROI can be indicated including the entire myocardium (labeled 2101) as shown in FIG. 21B. By evaluating the average, sum, median or some other metric of the pixel values within this ROI over time information about the rate of contrast outflow or inflow within this entire region is obtained. The time difference between the start of contrast outflow 2103 and end of outflow 2104 provides insight with respect to the rate at which contrast is passed through the entire myocardium. This time will be lower in a healthy myocardium having a low resistance. Also, the derivative of the downslope 2105 provides information about the contrast outflow and myocardium resistance. A similar approach can be applied to the upslope representing the inflow or the time difference between upslope and downslope. To be able to compare this time difference between patients optionally, the amount and injection rate of the contrast liquid can be standardized, or a normalization can be performed using the plot of FIG. 21A by normalizing for example based on the area under the curve. To determine the microvascular resistance locally, the region of interest can be narrowed to the region of interest.

### Artificial Intelligence

Another approach to determine an index for microvascular status is using artificial intelligence/deep learning. The artificial intelligence can use angiographic image data as input, but also additional information can be used. For example, projection angles of the angiographic images, angiographic images from multiple projection angles, patient information like age, weight, etc. but also clinical information/data like diabetes, hypertension etc.

Artificial intelligence can be used to calculate an index for microvascular status, or it can be used to calculate one or multiple of the before mentioned steps, e.g., determine volumetric flow rate 102, determine pressure drop 103 etc. or combinations.

### Alternative Indexes

Another index that provides information about the microvasculature is for example, a coronary flow reserve (CFR) index, which is quantitative data that represents the ratio of flow through a coronary artery with the patient in a rest state relative to flow through the coronary artery with the patient in an active/hyperemic state. In embodiments, this parameter can be derived by determining the coronary volumetric flow rate as described in step 102 on images acquired in the rest state of the patient and alternatively by images in rest state and the hyperemic state of the patient. An X-ray angiographic image acquisition in hyperemic state of the patient is performed by performing an X-ray acquisition of the coronary after inducing hyperemia for instance by intracoronary or intravenous administration of adenosine or papaverine as for instance described by De Bruyne et al. in “Intracoronary and intravenous adenosine 5′-triphosphate, adenosine, papaverine, and contrast medium to assess fractional flow reserve in humans”, Circulation. 2003; 107(14): 1877-1883. The fraction of the coronary volumetric flow rate derived from the X-ray angiographic image acquired at rest with respect to the coronary volumetric flow rate derived from an X-ray angiographic image at hyperemic state (or modelled from X-ray images acquired at rest state) gives the CFR index as illustrated in equation 25.

$$CFR = \frac{{Q}_{h y p}}{{Q}_{r e s t}} \qquad Eqn . ( 25 )$$

CFR=Coronary flow reserve
Q_{hyp}=coronary volumetric flow rate determined using step 102 at hyperemic patient state
Qᵣₑₛₜ=coronary volumetric flow rate determined using step 102 at hyperemic patient state

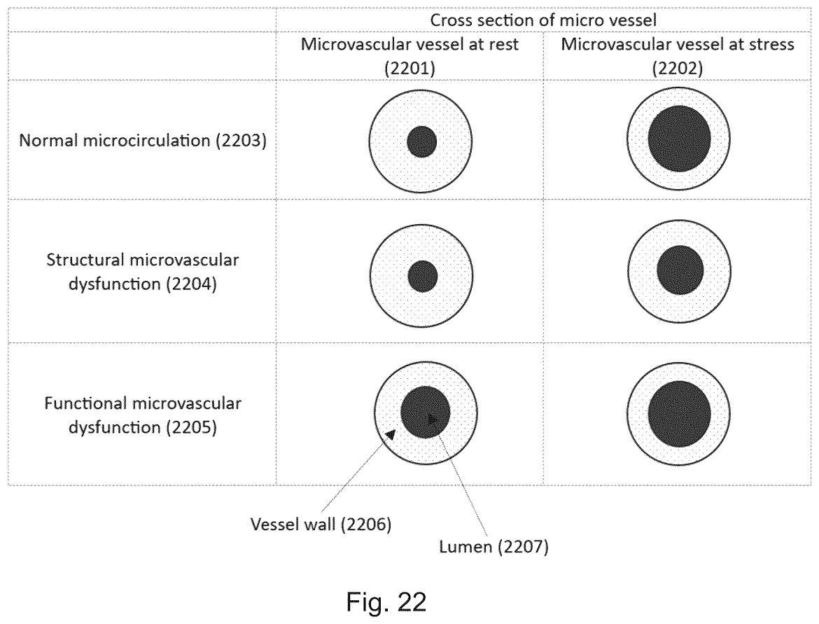

Another index that provides information about microvasculature dysfunction is the type of microvasculature dysfunction. There are two types of microvasculature dysfunction, structural microvasculature dysfunction and functional microvasculature dysfunction. Structural dysfunction involves physical alterations or abnormalities in the microvessels. This can include changes in vessel wall thickness, remodeling, or the presence of abnormalities such as fibrosis. Structural dysfunction microvasculature dysfunction can result from chronic inflammation, oxidative stress, and conditions like atherosclerosis. This leads to reduced blood flow, increased resistance, and impaired nutrient exchange. Functional microvasculature dysfunction refers to abnormalities in the dynamic regulation of blood flow and vessel responsiveness without necessarily involving physical changes in the vessel structure and can be caused by impaired vasodilation (inability of blood vessels to widen appropriately) or vasoconstriction (inability of blood vessels to constrict appropriately). FIG. 22 further shows the above differences between normal microcirculation (2203), structural microvasculature dysfunction (2204) and functional microvasculature dysfunction (2205). A cross sectional slice of the microvessel is shown of the aforementioned three situations and both in rest (2201) and in stress (2202). In normal microcirculation (2203) the vessel lumen (2207) at rest is narrowed and the diameter is sufficient to provide enough oxygen to the myocardium muscle. During stress, more oxygen is required, and the vascular tone of the vessel decreases, resulting in a widening of the vessel lumen. Vascular tone means the contractile activity of vascular smooth muscle cells in the walls (2206) of small arteries and arterioles. Within structural microvascular dysfunction (2204), the micro vessel lumen at rest behaves similarly as in normal microcirculation, but the vessel widening is limited during stress, resulting in a small lumen at stress with respect to a normal microcirculation. Meaning that in structural microvascular dysfunction, coronary blood flow and microvascular resistance at rest is normal, and coronary blood flow in stress is reduced and the microvascular resistance in stress is increased. Within functional microvascular dysfunction (2205), the micro vessel tone at rest is already decreased and during stress the micro vessel tone is generally similar or slightly decreased with respect to a normal microcirculation at stress. Meaning that in functional microvascular dysfunction, coronary blood flow at rest is normal (compensated by increased vessel lumen at rest) and microvascular resistance at rest is decreased, at stress the coronary blood flow is also reduced and the microvascular resistance in stress is similar or slightly increased as in normal microcirculation.

On order to distinguish between normal microcirculation (2203), structural microvasculature dysfunction (2204) and functional microvasculature dysfunction (2205) a threshold for coronary blood flow and/or microvascular resistance as calculated in accordance with the method described in this patent application based on an X-ray angiographic image sequence at rest and or an X-ray angiographic image sequence during hyperemia. The latter can also be based on an X-ray angiographic image sequence acquired at rest in which hyperemia is simulated or modelled in accordance with the methods described in this patent application. Several options can be followed to define these thresholds. For instance, based on the statistical difference in coronary blood flow and or microvascular resistance in a healthy population and a disease population (functional and/or structural microvascular dysfunction). The disease population and type of microvascular dysfunction can be identified by invasive IMR or invasive bolus thermodilution. Another approach to define the disease population is identifying an event after a predefined follow up period, for instance one year. The event can be defined as major adverse cardiovascular events, a composite of cardiovascular death, myocardial infarction, hospitalization for heart failure, or ischemia driven revascularization. Having the coronary blood flow as derived by the methods described by this patent application, the statistics involved to define the threshold can be lowest, middle, or highest tertile of the derived coronary blood flow and or microvascular resistance within the whole population (healthy and disease), or other statistical test to distinguish two groups.

Operations can be performed by processor unit on a standalone system, or a semi-standalone system which is connected to the X-ray cinefluorograpic system (FIG. 2) or any other image system to acquire two-dimensional angiographic image sequences.

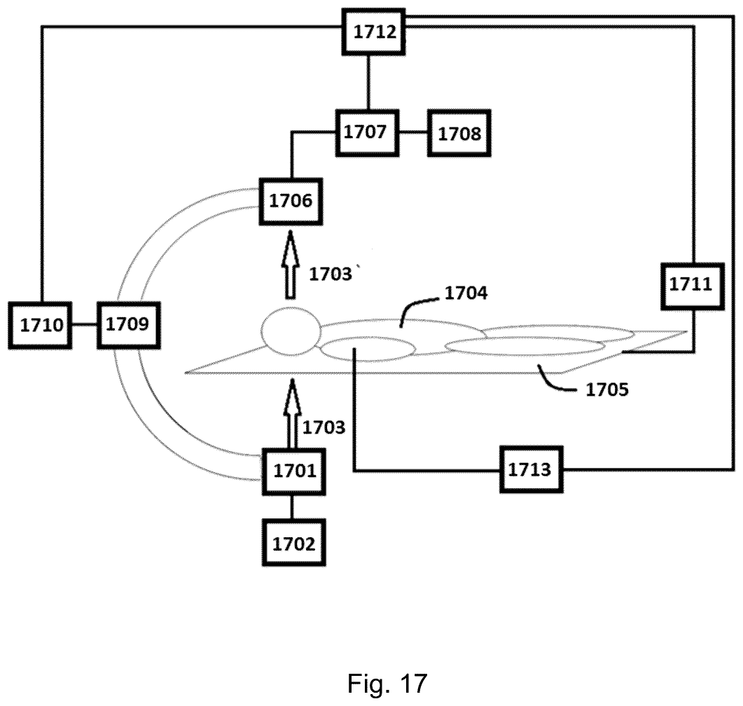

FIG. 17 illustrates an example of a high-level block diagram of an X-ray cinefluorograpic system. In this block diagram an example is shown on how embodiments could integrate in such a system.

Portions of the system (as defined by various functional blocks) may be implemented with dedicated hardware, analog and/or digital circuitry, and/or one or more processors operating program instructions stored in memory.

The X-ray system of FIG. 17 includes an X-ray tube 1701 with a high voltage generator 1702 that generates an X-ray beam 1703. The high voltage generator 1702 controls and delivers power to the X-ray tube 1701. The high voltage generator 1702 applies a high voltage across the vacuum gap between the cathode and the rotating anode of the X-ray tube 1701. Due to the voltage applied to the X-ray tube 1701, electron transfer occurs from the cathode to the anode of the X-ray tube 1701 resulting in X-ray photon-generating effect also called Bremsstrahlung. The generated photons form an X-ray beam 1703 directed to the image detector 1706.

An X-ray beam 1703 comprises of photons with a spectrum of energies that range up to a maximum determined by among others the voltage and current submitted to the X-ray tube 1701. The X-ray beam 1703 then passes through the patient 1704 that lies on an adjustable table 1705. The X-ray photons of the X-ray beam 1703 penetrate the tissue of the patient to a varying degree. Different structures in patient 1704 can absorb different fractions of the radiation, modulating the beam intensity. The modulated X-ray beam 1703′ that exits from patient 1704 is detected by the image detector 1706 that is located opposite of the X-ray tube. This image detector 1706 can either be an indirect or a direct detection system.

In the case of an indirect detection system, the image detector 1706 comprises of a vacuum tube (the X-ray image intensifier) that converts the X-ray exit beam 1703′ into an amplified visible light image. This amplified visible light image is then transmitted to a visible light image receptor such as a digital video camera for image display and recording. This results in a digital image signal.

In the case of a direct detection system, the image detector 1706 comprises of a flat panel detector. The flat panel detector directly converts the X-ray exit beam 1703′ into a digital image signal. The digital image signal resulting from the image detector 1706 is passed through a digital image processing unit 1707. The digital image processing unit 1707 converts the digital image signal from 1706 into a corrected X-ray image (for instance inverted and/or contrast enhanced) in a standard image file format for instance DICOM. The corrected X-ray image can then be stored on hard drive 1708.

Furthermore, the X-ray system of FIG. 17 comprises of a C-arm 1709. The C-arm holds the X-ray tube 1701 and the image detector 1706 in such a manner that the patient 1704 and the adjustable table 1705 lie between the X-ray tube 1701 and the image detector 1706. The C-arm can be moved (rotated and angulated) to a desired position to acquire a certain projection in a controlled manner using the C-arm control 1710. The C-arm control allows for manual or automatic input for adjustment of the C-arm in the desired position for the X-ray recording at a certain projection.

The X-ray system of FIG. 17 can either be a single plane or a bi-plane imaging system. In the case of a bi-plane imaging system, multiple C-arms 1709 are present each consisting of an X-ray tube 1701, an image detector 1706 and a C-arm control 1710.

Additionally, the adjustable table 1705 can be moved using the table control 1711. The adjustable table 1705 can be moved along the x, y and z axis as well as tilted around a certain point.

Furthermore, a measuring unit 1713 is present in the X-ray system. This measuring unit contains information regarding the patient, for instance information regarding ECG, aortic pressure, biomarkers, and/or height, length etc.

A general unit 1712 is also present in the X-ray system. This general unit 1712 can be used to interact with the C-arm control 1710, the table control 1711, the digital image processing unit 1707, and the measuring unit 1713.

An embodiment is implemented by the X-ray system of FIG. 17 as follows. A clinician or other user acquires at least two X-ray angiographic image sequences of a patient 1704 by using the C-arm control 1710 to move the C-arm 1709 to a desired position relative to the patient 1704. The patient 1704 lies on the adjustable table 1705 that has been moved by the user to a certain position using the table control 1711.

The X-ray image sequences are then generated using the high voltage generator 1702, the X-ray tube 1701, the image detector 1706 and the digital image processing unit 1707 as described above. These images are then stored on the hard drive 1708. Using these X-ray image sequences, the general processing unit 1712 performs the methods as described by present application, as for instance as described by FIG. 1 using the information of the measuring unit 1713, the digital image processing unit 1707, C-arm control unit 1710 and the table control unit 1711.

The information derived from the workflow as described herein, including one or more indices that characterize properties of microvasculature tissue (for example, the index for dysfunction or resistance in the microvascular tissue and/or or coronary flow reserve (CFR) index, can be presented for display on a display device, such as a display screen that is operably coupled to the general processing unit 1712 of FIG. 17.

There have been described and illustrated herein several embodiments of a method and apparatus for restoring missing information regarding the order and the flow direction of the velocity components. While particular embodiments of the invention have been described, it is not intended that the invention be limited thereto, as it is intended that the invention be as broad in scope as the art will allow and that the specification be read likewise. For example, the data processing operations can be performed offline on images stored in digital storage, such as a PACS commonly used in the medical imaging arts. It will therefore be appreciated by those skilled in the art that yet other modifications could be made to the provided invention without deviating from its spirit and scope as claimed.

The embodiments described herein may include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer, or speaker). Such a system may also include one or more storage devices, such as disk drives, optical storage devices and solid-state storage devices such as random-access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.) and working memory as described above. The computer-readable storage media reader can be connected with, or configured to receive, a computer-readable storage medium, representing remote, local, fixed and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

Preferred embodiments of this disclosure are described herein, including the best mode known to the inventors for carrying out the invention. Variations of those preferred embodiments may become apparent to those of ordinary skill in the art upon reading the foregoing description. The inventors expect skilled artisans to employ such variations as appropriate and the inventors intend for embodiments of the present disclosure to be practiced otherwise than as specifically described herein. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated herein or otherwise clearly contradicted by context.

All references, including publications, patent applications and patents, cited herein are hereby incorporated by reference to the same extent as if each reference were individually and specifically indicated to be incorporated by reference and were set forth in its entirety herein.

What is claimed is:

1. A computer-implemented method for characterizing a property of microvascular tissue that is supplied with blood via a coronary artery under investigation, the method comprising:
i) obtaining an x-ray angiographic image sequence of the coronary artery under investigation acquired while contrast agent flows into and through the coronary artery under investigation;
ii) using the angiographic image sequence of i) to determine a volumetric flow rate for flow through the coronary artery under investigation; and
iii) determining an index that represents a property of the microvascular tissue that is supplied with blood via the coronary artery under investigation based on the volumetric flow rate of ii).

2. A method according to claim 1, wherein:
the operations of i) to iii) are performed automatically by a processor without human input.

3. A method according to claim 1, wherein:
the volumetric flow rate is based on flow velocity of a contrast bolus front within the angiographic image sequence of i) and cross-sectional area of the coronary artery under investigation at multiple positions along the coronary artery under investigation within the angiographic image sequence of i).

4. A method according to claim 1, wherein:
the volumetric flow rate is based on propagation time of a contrast bolus front within the angiographic image sequence of i) and a vessel volume for the coronary artery of interest.

5. A method according to claim 4, wherein:
the vessel volume is determined from a 3D reconstruction of the coronary artery of interest.

6. A method according to claim 4, wherein:
the vessel volume is based on determining one or more diameters of the coronary artery of interest along the of the coronary artery of interest.

7. A method according to claim 1, wherein:
the flow velocity of the contrast bolus front is determined from distance that the contrast bolus front travels in the angiographic image sequence of i) as a function of time.

8. A method according to claim 1, wherein:
the flow velocity of the contrast bolus front is determined from image analysis of the angiographic image sequence of i), wherein the image analysis determines a proximal position for the coronary artery of interest, a proximal position for the coronary artery of interest, a vessel path extending along the coronary vessel of interest between the proximal position to the distal position, and propagation of the contrast bolus front along the vessel path.

9. A method according to claim 8, wherein:
at least one of the proximal position and the distal position is determined using artificial intelligence and/or deep learning techniques.

10. A method according to claim 9, wherein:
the artificial intelligence and/or deep learning techniques employ dichotomous image segmentation.

11. A method according to claim 9, wherein:
the artificial intelligence and/or deep learning techniques employ additional information selected from the groups consisting of vessel type, rotation and angulation used in image acquisition, ECG information, heart dominance information, and time between image frames.

12. A method according to claim 9, wherein:
the artificial intelligence and/or deep learning techniques employ a vesselness filter applied to multiple image frames of the angiographic image sequence of i).

13. A method according to claim 8, wherein:
the proximal position is determined from detection of position of a guiding catheter used for injection of the contrast agent into the coronary vessel of interest.

14. A method according to claim 8, wherein:
the vessel path is determined using a wave propagation algorithm between the proximal position and distal position.

15. A method according to claim 1, wherein:
the microvascular tissue is part of the myocardium.

16. A method according to claim 1, wherein:
the volumetric flow rate of ii) is characteristic of volumetric flow rate for part of a cardiac cycle.

17. A method according to claim 1, wherein:
the volumetric flow rate of ii) is characteristic of average flow velocity and average volumetric flow rate over a cardiac cycle.

18. A method according to claim 1, wherein:
the index comprises quantitative data that represents amount of dysfunction or resistance in the microvascular tissue that is supplied with blood via the coronary artery under investigation.

19. A method according to claim 18, further comprising:
determining a pressure drop associated with the coronary artery under investigation;
wherein the index of iii) is determined from the volumetric flow rate of ii) and the pressure drop.

20. A method according to claim 18, wherein:
the index of iii) is normalized based on at least one parameter selected from the group consisting of cardiac mass, coronary volume, coronary artery cross-sectional area, patient weight, height, body surface area (BSA) or body mass index (BMI), heart dominance, or combinations thereof.

21. A method according to claim 1, wherein:
the index comprises quantitative data that represents the ratio of flow through the coronary artery under investigation at rest relative to flow through the coronary artery under investigation in the hyperemic state.

22. A method according to claim 21, further comprising:
using the at least one angiographic image of i) to determine a first volumetric flow rate for flow through the coronary artery under investigation with the patient in a rest state;
using the at least one angiographic image of i) to determine a second volumetric flow rate for flow through the coronary artery under investigation with the patient in an active/hyperemic state; and
determining the index from the first and second volumetric flow rates.

23. A non-transitory computer readable medium, having stored thereon, instructions, which when executed by a computing device, cause the computing device to perform the method according claim 1.

24. An apparatus for acquiring an image data set of a patient, the apparatus comprising a data processing module configured to perform the method according to claim 1 to characterize a property of microvascular tissue that is supplied with blood via a coronary artery under investigation.
